SEQUENCE LISTING (14 sequences)

SEQ ID NO: 1 - protein, 20 aa, synthetic construct
  SITE 4: note = X is modified lysine with secondary amine and terminal azide
  SITE 6: note = X is modified lysine with secondary amine and terminal azide
  SITE 8: note = X is modified lysine with secondary amine and terminal azide
  SITE 13: note = X is modified lysine with secondary amine and terminal azide
  SITE 17: note = X is modified lysine with secondary amine and terminal azide
VLTXVXTXVPPTXVQVXVFV

SEQ ID NO: 2 - protein, 20 aa, synthetic construct
  SITE 2: note = X is modified lysine with secondary amine and terminal azide
  SITE 4: note = X is modified lysine with secondary amine and terminal azide
  SITE 17: note = X is modified lysine with secondary amine and terminal azide
  SITE 19: note = X is modified lysine with secondary amine and terminal azide
VXTXVEVEVPPTEVETXVXV

SEQ ID NO: 3 - protein, 20 aa, synthetic construct
  SITE 4: note = X is modified lysine with secondary amine and terminal azide
  SITE 8: note = X is modified lysine with secondary amine and terminal azide
  SITE 15: note = X is modified lysine with secondary amine and terminal azide
  SITE 19: note = X is modified lysine with secondary amine and terminal azide
VETXVEVXVPPTEVXTEVXV

SEQ ID NO: 4 - protein, 20 aa, synthetic construct
  SITE 2: note = X is modified lysine with secondary amine and terminal azide
  SITE 4: note = X is modified lysine with secondary amine and terminal azide
  SITE 6: note = X is modified lysine with secondary amine and terminal azide
  SITE 8: note = X is modified lysine with secondary amine and terminal azide
  SITE 13: note = X is modified lysine with secondary amine and terminal azide
  SITE 15: note = X is modified lysine with secondary amine and terminal azide
  SITE 17: note = X is modified lysine with secondary amine and terminal azide
  SITE 19: note = X is modified lysine with secondary amine and terminal azide
VXVXVXVXVPPTXVXVXVXV

SEQ ID NO: 5 - protein, 20 aa, synthetic construct
  SITE 2: note = X is modified lysine with secondary amine and terminal azide
  SITE 4: note = X is modified lysine with secondary amine and terminal azide
  SITE 6: note = X is modified lysine with secondary amine and terminal azide
  SITE 8: note = X is modified lysine with secondary amine and terminal azide
  SITE 13: note = X is modified lysine with secondary amine and terminal azide
  SITE 17: note = X is modified lysine with secondary amine and terminal azide
  SITE 19: note = X is modified lysine with secondary amine and terminal azide
VXVXVXVXVPPTXVEVXVXV

SEQ ID NO: 6-8 - skipped (3 entries)

SEQ ID NO: 9 - protein, 8 aa, synthetic construct
  SITE 7: note = X is modified lysine with secondary amine and terminal azide
YGGASVXA

SEQ ID NO: 10 - protein, 8 aa, synthetic construct
YGGASVGA

SEQ ID NO: 11 - protein, 8 aa, synthetic construct
  SITE 7: note = X is modified lysine with terminal azide
YGGASVXA

SEQ ID NO: 12 - protein, 8 aa, synthetic construct
YGGASVKA

SEQ ID NO: 13 - protein, 20 aa, synthetic construct
  SITE 2: note = X is modified lysine with terminal azide
  SITE 4: note = X is modified lysine with terminal azide
  SITE 6: note = X is modified lysine with terminal azide
  SITE 8: note = X is modified lysine with terminal azide
  SITE 13: note = X is modified lysine with terminal azide
  SITE 17: note = X is modified lysine with terminal azide
  SITE 19: note = X is modified lysine with terminal azide
VXVXVXVXVPPTXVEVXVXV

SEQ ID NO: 14 - protein, 20 aa, synthetic construct
VKVKVKVKVPPTKVEVKVKV

(19) United States
(12) Patent Application Publication
(10) Pub. No.: US 2025/0002535 A1
(43) Pub. Date: Jan. 2, 2025

(54) PEPTIDE HYDROGEL COMPRISING HYDROPHILIC AZIDE-CONTAINING AMINO ACID AND ITS USE

(71) Applicant: The United States of America, as represented by the Secretary, Department of Health and Human servi, Bethesda, MD (US)

(72) Inventors: Joel Schneider, Frederick, MD (US); Yixin Xie, Frederick, MD (US); Tania Lopez-Silva, Rockville, MD (US)

(73) Assignee: The United States of America, as represented by the Secretary, Department of Health and Human servi, (omitted)

(21) Appl. No.: 18/340,863

(22) Filed: Jun. 25, 2023

(51) Int. Cl. C07K 7/08; C08J 3/075

(52) U.S. Cl. CPC C07K 7/08; C08J 3/075; C08J 2389/00

(57) ABSTRACT

Provided herein is a novel β-hairpin peptide hydrogel that can be labeled with contrast agent for CECT imaging. The β-hairpin peptide hydrogel undergoes shear-thinning upon application of shear stress, and rheological recovery upon removal of the shear stress, and therefore is well-suited for delivery by syringe to a target location in a subject. Once delivered, the labeled β-hairpin peptide hydrogel remains in place to allow CECT imaging of the target location in the subject, which can be used to guide administration of further agents to the target location.

### STATEMENT REGARDING FEDERALLY SPONSORED RESEARCH AND DEVELOPMENT

This invention was made with Government Support under project number ZIABC011313-12 awarded by the National Institutes of Health, National Cancer Institute. The Government has certain rights in this invention.

### FIELD OF THE DISCLOSURE

This relates to peptide hydrogels containing contrast agents for contrast enhanced computed tomography (CECT) imaging that can undergo gel-to-solution (gel-sol) and solution-to-gel (sol-gel) phase transitions to facilitate local delivery to a target location in a subject.

### SEQUENCE LISTING

This application contains a Sequence Listing submitted as an XML file in the form of the file named “Sequence_Listing_4239_109373_01”, having a size of 26,262 bytes, and created on Oct. 5, 2023. The information contained in this electronic file is hereby incorporated by reference in its entirety pursuant to 37 CFR § 1.52(e)(5).

### BACKGROUND

Contrast agents are provided to patients to improve the ability to detect blood vessels and organs using contrast enhanced computed tomography (CECT) imaging. CECT imaging of a target location in the patent is performed while the contrast agent is administered systemically and then disperses through the patient. However, available methods and reagents lack the capacity for locally delivered and retained contrast agent to enable CECT imaging of a target location over longer periods of time and to guide administration of heterologous agents to the target location.

### SUMMARY

Provided herein is a novel β-hairpin peptide hydrogel that can be labeled with contrast agent for CECT imaging. The β-hairpin peptide hydrogel undergoes shear-thinning upon application of shear stress, and rheological recovery upon removal of the shear stress, and therefore is well-suited for delivery by syringe to a target location in a subject. Once delivered, the labeled β-hairpin peptide hydrogel remains in place to allow CECT imaging of the target location in the subject, which can be used to guide administration of further agents to the target location.

In some aspects, a peptide hydrogel is provided that is formed from an amphiphilic cationic β-hairpin peptide. The amphiphilic cationic β-hairpin peptide comprises an unnatural lysine

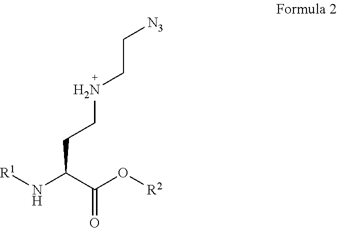

wherein R¹ is another amino acid in the peptide or the N-terminus of the peptide and R² is another amino acid in the peptide or the C-terminus of the peptide. In some aspects, the amphiphilic cationic β-hairpin peptide in the peptide hydrogel comprises an amino acid sequence set forth as:

| (SEQ ID NO: 6) | |
|---|---|
| | (XZ)ₙ X^{D}PPX (ZX)ₙ; |
| (SEQ ID NO: 7) | |
| | (XZ)ₙ X^{D}PGX (ZX)ₙ; |
| | or |
| (SEQ ID NO: 8) | |
| | (XZ)ₙ XNGX (ZX)ₙ; |

and
wherein
each X is individually selected from V, I, L, M, T, F, W, and Y;
at least one Z is the unnatural lysine and the remaining Z are individually selected from any amino acid;
X^{D} is a D-amino acid selected from a V, I, L, M, T, F, W, and Y D-amino acid; and
n is from 3 to 5.

In some aspects, the amphiphilic cationic β-hairpin peptide in the peptide hydrogel comprises or consists of an amino acid sequence set forth as any one of

| (SEQ ID NO: 1) | |
|---|---|
| | VLTZVZTZV^{D}PPTZVQVZVFV; |
| (SEQ ID NO: 2) | |
| | VZTZVEVEV^{D}PPTEVETZVZV; |
| (SEQ ID NO: 3) | |
| | VETZVEVZV^{D}PPTEVZTEVZV; |
| (SEQ ID NO: 4) | |
| | VZVZVZVZV^{D}PPTZVZVZVZV; |
| | and |
| (SEQ ID NO: 5) | |
| | VZVZVZVZV^{D}PPTZVEVZVZV; |

and
wherein each Z is the unnatural lysine.

In some aspects, the amphiphilic cationic β-hairpin peptide in the peptide hydrogel is acetylated at the N-terminus, amidated at the C-terminus, or acetylated at the N-terminus and amidated at the C-terminus.

In some aspects, the amphiphilic cationic β-hairpin peptide comprises or consists of:

| | XP1 |
|---|---|
| (SEQ ID NO: 1) | |
| | Ac-VLTZVZTZV^{D}PPTZVQVZVFV-NH₂ |
| | XP2 |
| (SEQ ID NO: 2) | |
| | VZTZVEVEV^{D}PPTEVETZVZV-NH₂ |
| | XP3 |
| (SEQ ID NO: 3) | |
| | VETZVEVZV^{D}PPTEVZTEVZV-NH₂ |
| | XP4 |
| (SEQ ID NO: 3) | |
| | VETZVEVZV^{D}PPTEVZTEVZV |
| | XP5 |
| (SEQ ID NO: 2) | |
| | Ac-VZTZVEVEV^{D}PPTEVETZVZV |
| | XP6 |
| (SEQ ID NO: 3) | |
| | Ac-VETZVEVZV^{D}PPTEVZTEVZV |
| | XP7 |
| (SEQ ID NO: 4) | |
| | VZVZVZVZV^{D}PPTZVZVZVZV-NH₂ |
| | XP8 |
| (SEQ ID NO: 5) | |
| | VZVZVZVZV^{D}PPTZVEVZVZV-NH₂ |

wherein each Z is the unnatural lysine, Ac indicates that the peptide is acetylated at the N-terminus, and NH₂ indicates that the peptide is amidated at the C-terminus.

In some aspects, one or more of the unnatural lysine residues in the amphiphilic cationic (β-hairpin peptide in the peptide hydrogel are linked to a detectable marker by Strain Promoted Azide-Alkyne Cycloaddition (SPAAC) chemistry via the terminal azide group of the unnatural lysine. In some aspects, the detectable marker is a contrast agent for contrast enhanced computed tomography (CECT) imaging, for example, a contrast agent comprising a triiodo group. In some aspects, the contrast agent for CECT linked to the unnatural lysine in the amphiphilic cationic β-hairpin peptide is selected from any one of Triiodobenzoic acid (TIBA), iohexol, iopromide, iothalamate, ioxaglate, and iodixanol. In some aspects, the contrast agent for CECT comprises any one of:

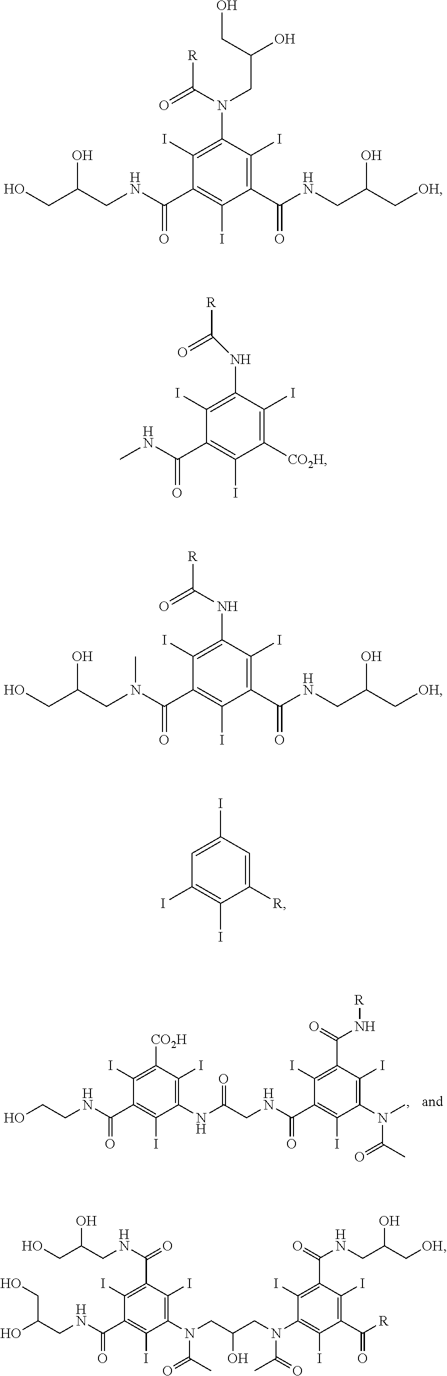

wherein R is a linkage to the unnatural lysine residues in the amphiphilic cationic β-hairpin peptide by SPAAC chemistry.

In some aspects, a syringe is provided that contains the peptide hydrogel provided herein.

Also, provided are methods of making the peptide hydrogel provided herein, as well as methods of use. In some aspects, a method of contrast enhanced computed tomography (CECT) imaging of a subject is provided, the method comprising administering a peptide hydrogel linked to a detectable marker for CECT imaging as provided herein to a target location in the subject, and conducting a contrast CT scan of the target location in the subject.

The foregoing and other features and advantages of this disclosure will become more apparent from the following detailed description of several embodiments which proceeds with reference to the accompanying figures.

### BRIEF DESCRIPTION OF THE FIGURES

FIG. 1. Peptides incorporating residue 1 exploring scope of Fmoc-based SPPS.

FIGS. 2A-2B. (FIG. 2A) RP-HPLC elution profiles of peptides 12, 13, control 14 and Gly-standard 15. Inset: retention times and Δt_{R(Gly)} values. (FIG. 2B) UV-vis spectra of peptide solutions in water before (solid line) and after (dash line) centrifugation. Peptides 16 and 18 at 20 mg/mL 1:300 dilutions, and peptide 17 at 1 mg/mL.

FIGS. 3A-3F. Reaction kinetics of residue 1 and ^{N3}K with DBCO and BCN. (FIG. 3A) Ligation of peptide 12 with DBCO in HBS buffer at pH 7.4 and 25° C. (FIG. 3B) Second-order rate constant (k₂) determination of peptide 12 or (FIG. 3C) peptide 13 with DBCO calculated based on the k_{obs} across different peptide concentrations. k_{obs} was calculated using pseudo-first order kinetics conditions by monitoring the absorbance decay at 308 nm. (FIG. 3D) Ligation of compound 2 with BCN in MeOD at 25° C. (FIG. 3E) Second-order rate constant (k₂) determination of 2 or (FIG. 3F) Fmoc-^{N3}K calculated by linear regression between k2t and time. k₂t was calculated under second order kinetics conditions by monitoring the concentration of product using NMR and equation S1.

FIGS. 4A-4B. ESI (+) mass spectra of SPAAC reactions between peptide 16 and different BCN equivalents at (FIG. 4A) room temperature and (FIG. 4B) 37° C. For clarity, only the (M+5H)⁵⁺ charge state manifold is shown. Complete spectra are provided in FIGS. 22-23.

FIG. 5A-5C. Structure of peptides 6-18. Peptide purity was determined by C18 RP-HPLC monitoring at 220 nm wavelength. X represents residue 1. ^{a}Coupling: 4 equiv. Fmoc-protected amino acid, 4 equiv. HATU, 6 equiv. DIPEA, DMF, r.t. 0.5 h. ^{b}Coupling condition: 5 equiv. HOAt, 5 equiv. DIC in DMF, r.t. 1 h. ^{c}Coupling condition: 5 equiv PyBOP, 10 equiv DIPEA in DMF, r.t. 1 h. ^{d}Coupling condition: 5 equiv. Oxyma, 5 equiv. DIC, DMF, microwave 90° C. 4 min.

FIG. 6. RP-HPLC trace of peptide 16 (N3NHMAX8) and MAX8 (Peptide 18). Peptide 16 eluted at 34% STD B and MAX8 eluted at 35% STD B.

FIG. 7A-7B. Pseudo-first order kinetics of peptide 12 and DBCO in HBS. The plot shows the absorbance decay at 308 nm measured by UV-vis spectrophotometer (black curve). The nonlinear regression calculation by Prism software is fitted as red curve. The second-order rate constant k₂ was calculated based on plot of k_{obs} across four different concentrations of peptide 12 using Prism software.

FIG. 8A-8B. Pseudo-first order kinetics of peptide 13 and DBCO in HBS. The plot shows the absorbance decay at 308 nm measured by UV-vis spectrophotometer (black curve). The nonlinear regression calculation by Prism software is fitted as red curve. The second-order rate constant k₂ was calculated based on plot of k_{obs} across four different concentrations of peptide 13 using Prism software.

FIG. 9. Second order kinetics of 2 and BCN in MeOH. In the NMR spectrum, the integration of 2.65 ppm peak (using 3.64-3.40 ppm as internal standard) was used to calculate the concentration of product ([P]) at each time point. k₂t was calculated based on equation S1. The second-order rate constant k₂ was calculated by linear regression between k₂t and time.

FIG. 10. Second order kinetics of Fmoc azido lysine and BCN in MeOH. In the NMR spectrum, the integration of 2.91 ppm peak (using 3.64 ppm as internal standard) was used to calculate the concentration of product ([P]) at each time point. k₂t was calculated based on equation S1. The second-order rate constant k₂ was calculated by linear regression between k2t and time.

FIG. 11. ¹H NMR of compound 4.

FIG. 12. ¹³C NMR of compound 4.

FIG. 13. ¹H NMR of compound 3a

FIG. 14. ¹³C NMR of compound 3a

FIG. 15. ¹H NMR of compound 2

FIG. 16. ¹³C NMR of compound 2

FIG. 17. HSQC NMR of compound 2

FIGS. 18A-18B. HPLC trace and ESI (+) mass spectra of crude tripeptides 6 and 7 containing amino acid 1 (X). (FIG. 18A) XIA eluted at 11% STD B. (FIG. 18B) XPA eluted at 5% STD B.

FIGS. 19A-19D. HPLC trace and ESI (+) mass spectra of crude tetrapeptides 8-11 containing amino acid 1 (X). FSXV eluted at 15% STD B, FQXV eluted at 16% STD B, FRXV eluted at 16% STD B, and FHXV eluted at 16% STD B.

FIG. 20. ESI (+) mass spectra of peptides 12-15.

FIG. 21. HPLC trace and ESI (+) mass spectra of peptide 16 (N3NHMAX8) containing seven amino acid 1 (X). Peptide eluted at 34% STD B.

FIGS. 22A-22B. Mass spectra of peptide 16 and BCN reaction mixture after 24 h at room temperature at 1.1, 1.2, 1.5, and 2 equivalents of BCN per amino acid 1.

FIGS. 23A-23B. Mass spectra of peptide 16 and BCN reaction mixture after 24 h at 37° C. at 1.05, 1.1, 1.2, and 1.5 equivalents of BCN per amino acid 1.

FIG. 24. CD measurement of XP8 peptide. Temperature dependent CD spectroscopy indicates the β-sheet formation of 150 uM peptide in 1×HBS occurs with Tgel=18° C.

FIG. 25. Mechanical properties of the XP8 peptide hydrogel. Oscillatory rheology assays show that XP8 peptide hydrogel undergoes gelation in physiological buffer, and has shear-thinning recovery properties.

FIGS. 26 and 27. Overview of triiodo-labeling of β-hairpin peptide hydrogel containing unnatural lysine as provided herein.

FIG. 28. DBCO- and BCN-containing triiodo derivatives for SPAAC labeling of the peptide hydrogel formed with a β-hairpin peptide containing the unnatural lysine described herein.

FIG. 29. Examples of triiodo-containing groups for labeling of the peptide hydrogel formed with a β-hairpin peptide containing the unnatural lysine described herein.

FIG. 30. SPAAC ligation of a DBCO-containing triiodo derivative to a short peptide containing the azido-lysine described herein.

FIG. 31. SPAAC ligation of BCN to azido-MAX8 peptide. LCMS analysis demonstrated that a seven azido groups in azido-MAX8 are modified by BCN. azido-MAX8 peptide comprises SEQ ID NO: 5.

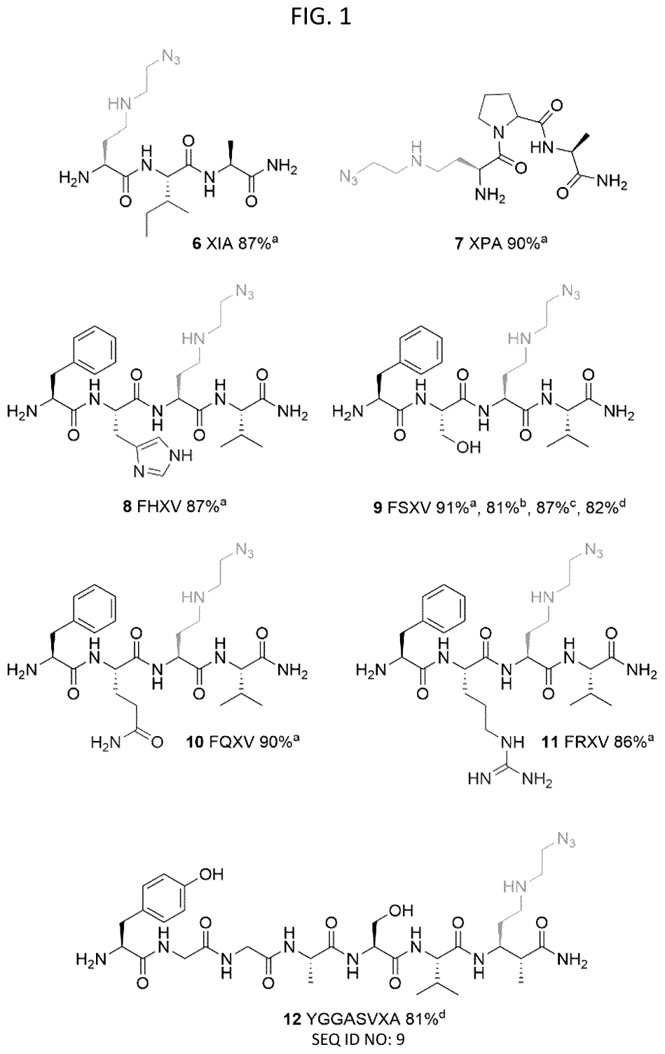

### DETAILED DESCRIPTION

### I. Introduction

Contrast agents are provided to patients to improve the ability to detect blood vessels and organs using contrast enhanced computed tomography (CECT) imaging. CECT imaging of a target location in the patent is performed while the contrast agent is administered systemically and then disperses through the patient. However, available methods and reagents lack the capacity for locally delivered and retained contrast agent to enable CECT imaging of a target location over longer periods of time and to guide administration of heterologous agents to the target location.

Provided herein is a novel β-hairpin peptide hydrogel that can be labeled with contrast agent for CECT imaging. The β-hairpin peptide hydrogel undergoes shear-thinning upon application of shear stress, and rheological recovery upon removal of the shear stress, and therefore is well-suited for delivery by syringe to a target location in a subject. Once delivered, the labeled β-hairpin peptide hydrogel remains in place to allow CECT imaging of the target location in the subject, which can be used to guide administration of further agents to the target location.

To generate the novel β-hairpin peptide hydrogel, a novel unnatural azido-lysine for use in Strain-Promoted Azide Alkyne Cycloaddition (SPAAC) ligation was designed and produced.

Incorporating ligation handles into macromolecules can dramatically increase their hydrophobicity affecting solubility and aggregation propensity. This is a significant problem that can negatively impact discovery projects as well as the development of biologics where solubility can become an issue in scaled preparation and formulation. The novel unnatural azido-lysine reported here overcomes this problem by having both a secondary amine and an azide in its side chain to impart charge and enhance solubility while allowing efficient click chemistry reactivity. In contrast, the commonly used azido-lysine, which does not have both a secondary amine and an azide in its side chain, suffers from hydrophobicity and low solubility problems.

The novel unnatural azido-lysine was incorporated into amphiphilic β-hairpin peptides, which are shown to adopt a β-hairpin conformation and to self-assemble to afford a fibrillar network in pH 7.4 buffer, becoming a mechanically rigid hydrogel with shear-thinning properties sufficient for syringe delivery. The novel azido-Lys residue in the hydrogel facilitates SPAAC ligation between iodinated bicyclononyne (BCN) and the azide-containing peptide, to produce a high mole percentage iodinated gel. The iodinated hydrogel can be injected into tumors for CT-imaging applications.

### II. Summary of Terms

The following explanations of terms and abbreviations are provided to better describe the present disclosure and to guide those of ordinary skill in the art in the practice of the present disclosure. As used herein, the singular forms “a,” “an,” and “the,” refer to both the singular as well as plural, unless the context clearly indicates otherwise. For example, the term “a peptide” includes single or plural peptides and can be considered equivalent to the phrase “at least one peptide.” As used herein, the term “comprises” means “includes.” Thus, “comprising a peptide” means “including a peptide” without excluding other elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing implementations from discussed prior art, the implementation numbers are not approximates unless the word “about” is recited. It is further to be understood that all base sizes or amino acid sizes, and all molecular weight or molecular mass values, given for nucleic acids or polypeptides are approximate, and are provided for description.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

A person of ordinary skill in the art would recognize that the definitions provided below are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). The presently disclosed compounds also include all isotopes of atoms present in the compounds, which can include, but are not limited to, deuterium, tritium, ¹⁸F, ¹⁴C, etc.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. Mixtures of different isomeric forms, including mixtures of enantiomers and/or stereoisomers, can be separated to provide each separate enantiomers and/or stereoisomer using techniques known to those of ordinary skill in the art, particularly with the benefit of the present disclosure. In cases of limited rotation, e.g., around the amide bond or between two directly attached rings such as pyridinyl rings, biphenyl groups, and the like, atropisomers are also possible and are also specifically included in the compounds disclosed herein.

In any implementations, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, methyl refers to both CH₃ or CH₃ wherein from 1 to 3 hydrogens are replaced by deuterium, such as in CDₓH₃₋ₓ.

As used herein, the term “substituted” refers to all subsequent modifiers in a term, for example in the term “substituted aliphatic-aromatic,” substitution may occur on the “aliphatic” portion, the “aromatic” portion or both portions of the aliphatic-aromatic group.

“Substituted,” when used to modify a specified group or moiety, means that at least one, and perhaps two or more, hydrogen atoms of the specified group or moiety is independently replaced with the same or different substituent groups. In a particular implementation, a group, moiety, or substituent may be substituted or unsubstituted, unless expressly defined as either “unsubstituted” or “substituted.” Accordingly, any of the functional groups specified herein may be unsubstituted or substituted unless the context indicates otherwise, or a particular structural formula precludes substitution. In particular implementations, a substituent may or may not be expressly defined as substituted but is still contemplated to be optionally substituted. For example, an “aliphatic” or a “cyclic” moiety may be unsubstituted or substituted, but an “unsubstituted aliphatic” or an “unsubstituted cyclic” is not substituted. In one implementation, a group that is substituted has at least one substituent up to the number of substituents possible for a particular moiety, such as 1 substituent, 2 substituents, 3 substituents, or 4 substituents.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

In order to facilitate review of the various implementations of the disclosure, the following explanations of specific terms are provided:

About: Unless context indicated otherwise, “about” refers to plus or minus 5% of a reference value. For example, “about” 100 refers to 95 to 105.

Agent: Any substance or any combination of substances that is useful for achieving an end or result; for example, a substance or combination of substances useful for decreasing or reducing tumor growth in a subject. Agents include effector molecules and detectable markers. In some implementations, the agent is a chemotherapeutic agent. The skilled artisan will understand that particular agents may be useful to achieve more than one result; for example, an agent may be useful as both a detectable marker and a chemotherapeutic agent.

Amphiphilic cationic β-hairpin peptide: A peptide that has a positive electrostatic charge at neutral pH and folds into a β-hairpin conformation under suitable conditions, such as when dissolved at 2.0% w/v in 50 mM Bis Tris Propane, pH 7.4, 150 mM NaCl, at 25° C. When folded into the β-hairpin conformation, one face of the hairpin is primarily hydrophobic, and the other is primarily hydrophilic. A non-limiting example of an amphiphilic cationic β-hairpin peptide is provided herein as azido-MAX8 peptide.

β-hairpin conformation: A structural conformation of a peptide or protein. The β-hairpin conformation includes two β-strands linked by a β-turn to form a “hairpin”-like shape. The structure is amphiphilic; thus, one face of the hairpin is primarily hydrophobic, and the other is primarily hydrophilic. A limited number of the side chains of hydrophobic amino acids can exist on the hydrophilic face of the hairpin and vice versa, but not so many as to change the overall amphiphilicity of the folded structure. A non-limiting example of a peptide that can fold into an β-hairpin conformation is provided herein as azido-MAX8.

Cationic amphiphilic peptide: A peptide that has a positive electrostatic charge at neutral pH and folds into a β-hairpin conformation under suitable conditions, such as when dissolved at 2.0% w/v in 50 mM Bis Tris Propane, pH 7.4, 150 mM NaCl, at 25° C. When folded into the β-hairpin conformation, one face of the hairpin is primarily hydrophobic, and the other is primarily hydrophilic. A non-limiting example of an amphiphilic cationic β-hairpin peptide is provided herein as the azido-MAX8 peptide.

Effective amount: An amount of an agent that is sufficient to produce a desired response, such as reducing or inhibiting one or more signs or symptoms associated with a condition or disease. In some examples, an “effective amount” is an amount that inhibits or prevents allograft rejection. In some examples, an “effective amount” is a therapeutically effective amount in which the agent alone or with one or more additional therapies, induces the desired response, such as an increase in survival, such as an increase in survival of 3 months, 6 months, 12 months, two years, or five years or longer.

Fluorenylmethoxycarbonyl protecting group (Fmoc): A protecting group used in organic synthesis and having the structure set forth as:

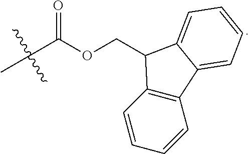 [SMILES: *C(=O)OCC1c2ccccc2-c2ccccc21]

Peptide: A chain of amino acids, typically less than 75 amino acids in length, such as 20-50 amino acids in length. The residues in a peptide can include post-translational or secondary modifications, such as glycosylation, sulfation or phosphorylation, as well as chemical modifications. “Peptide” applies to naturally occurring amino acid polymers and non-naturally occurring amino acid polymers, including amino acid polymers in which one or more amino acid residues are non-natural amino acids. A “residue” refers to an amino acid or amino acid mimetic incorporated in a peptide by an amide bond or amide bond mimetic. A peptide has an amino terminal (N-terminal) end and a carboxy terminal (C-terminal) end.

Typically, the amino acids making up a peptide are numbered in order, starting at the amino terminus and increasing in the direction toward the carboxy terminus of the peptide. Thus, when one amino acid is said to “follow” another, that amino acid is positioned closer to the carboxy terminal end of the peptide than the preceding amino acid.

Peptide hydrogel: A colloid gel including an internal phase and a dispersion medium, in which an aqueous solution is the dispersion medium and a self-assembled network of peptides is the internal phase. The peptides in the hydrogel are self-assembled and are folded into an β-hairpin conformation in the fibrillar network that forms the internal phase of the hydrogel. The peptide hydrogels disclosed herein are made using peptides that form an β-hairpin conformation in an aqueous solution comprising 150 mM NaCl and a pH of 7.4 at 25-37° C. Thus, an aqueous solution containing 2% w/v of a disclosed peptide and 150 mM NaCl and a pH of 7.4 forms a peptide hydrogel comprising a fibrillar network of the peptide when incubated at 25-37° C. in a container. Peptide hydrogels include a sufficient elastic modulus or stiffness that allows them to maintain shape. In several embodiments, the peptide hydrogel has an elastic modulus of 40 Pascal or greater. Peptide hydrogels formed from the disclosed self-assembled peptides in an β-hairpin conformation can be characterized by shear-thin/recovery rheological properties. The hydrogel undergoes a gel-sol phase transition upon application of shear stress, and a sol-gel phase transition upon removal of the shear stress. Thus, application of shear stress converts the solid-like gel into a viscous gel capable of flow, and cessation of the shear results in gel recovery. General information concerning peptide hydrogels having shear-thin/recovery rheological properties and methods of making same is provided, for example, in Sathaye, et al. Biomacromolecules, 2014, 15(11):3891-3900; Hule et al., 2008, Faraday Discuss, 139:251-420. In several embodiments, the peptide hydrogel can be a sterile hydrogel prepared with physiological and non-toxic dispersion medium for use to deliver therapeutics to a subject.

Polypeptide and peptide modifications: The present disclosure includes synthetic peptides, as well as derivatives (chemically functionalized polypeptide molecules obtained starting with the disclosed polypeptide sequences) and variants (homologs) of peptides described herein. The peptides disclosed herein include a sequence of amino acids that can include L- and/or D-amino acids, naturally occurring and otherwise.

Peptides can be modified by a variety of chemical techniques to produce derivatives having essentially the same activity as the unmodified polypeptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the polypeptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the polypeptide side chains can be converted to C₁-C₁₆ alkoxy or to a C₁-C₁₆ ester using well-recognized techniques. Phenyl and phenolic rings of the polypeptide side chains can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the polypeptide side chains can be extended to homologous C₂-C₄ alkylenes. Thiols can be protected with any one of a number of well-recognized protecting groups, such as acetamide groups. Those skilled in the art will also recognize methods for introducing cyclic structures into the polypeptides of this disclosure to select and provide conformational constraints to the structure that result in enhanced stability. For example, a C- or N-terminal cysteine can be added to the polypeptide, so that when oxidized the polypeptide will contain a disulfide bond, generating a cyclic polypeptide. Other polypeptide cyclizing methods include the formation of thioethers and carboxyl- and amino-terminal amides and esters.

Pharmaceutical composition: A composition including an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

Pharmaceutically acceptable carrier: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular implementations, suitable for administration to a subject the carrier may be sterile, and/or suspended or otherwise contained in a unit dosage form containing one or more measured doses of the composition suitable to induce the desired tumor response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Pharmaceutically acceptable salt or ester: Salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like.

Pharmaceutically acceptable salts of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of pharmacologically acceptable salts, see Berge et al., J. Pharm. Sci. 66:1 (1977).

Pharmaceutically acceptable esters include those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term addition salt as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Subject: Living multi-cellular vertebrate organisms, a category that includes human and non-human mammals. In one example, a subject is a human.

Strain Promoted Azide-Alkyne Cycloaddition (SPAAC): A type of chemical reaction tailored to generate covalent bonds quickly and reliably by joining small units comprising reactive groups together. A non-limiting example involves a 1,3-dipolar cycloaddition reaction between an azide functional group on a first reactant and an alkyne functional group on a second reactant. In SPAAC reactions, the alkyne is introduced in a strained difluorooctyne (DIFO), in which the electron-withdrawing, propargylic, gem-fluorines act together with the ring strain to greatly destabilize the alkyne. This destabilization increases the reaction driving force, and the desire of the cycloalkyne to relieve its ring strain. Other suitable SPAAC chemistry functional groups are known to those of skill in the art (see, e.g., Lahann (Ed), Click Chemistry for Biotechnology and Materials Science. Wiley, 2009, incorporated by reference herein in its entirety).

tert-butyloxycarbonyl protecting (Boc): A protecting group used in organic synthesis and having the structure set forth as:

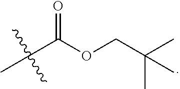 [SMILES: *C(=O)OCC(C)(C)C]

Under conditions sufficient for: A phrase that is used to describe any environment that permits a desired activity. In one example the desired activity is treatment of a tumor. In another example, the desired activity is a click chemistry reaction.

### III. Novel Unnatural Lysine and Polypeptides Comprising Same

Disclosed herein is an unnatural lysine amino acid for use in Strain-Promoted Azide Alkyne Cycloaddition (SPAAC) ligation having both a secondary amine and an azide in its side chain to impart charge and enhance solubility while allowing efficient click chemistry reactivity.

In some implementations, the unnatural lysine residue, or a stereoisomer, tautomer, or pharmaceutically acceptable salt or ester thereof, has a structure according to Formula I:

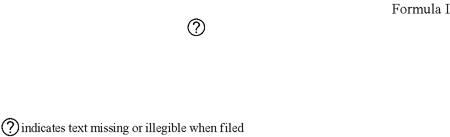

wherein X¹ is a protecting group and X² is a protecting group. In some implementations, X¹ and X² are independently selected from fluorenylmethoxycarbonyl protecting group (Fmoc), tert-butyloxycarbonyl protecting (Boc), and (S)-2-((((9H-fluoren-9-yl)methoxy)carbonyl)amino)-4-((2-azidoethyl)((2-(trimethylsilyl)ethoxy)carbonyl)amino)butanoic acid (Teoc). For example, X¹ is Fmoc and X² is Boc. In another example, For example, X¹ is Fmoc and X² is Teoc. In some implementations, the unnatural lysine residue, or a stereoisomer, tautomer, or pharmaceutically acceptable salt or ester thereof, has structure 2.

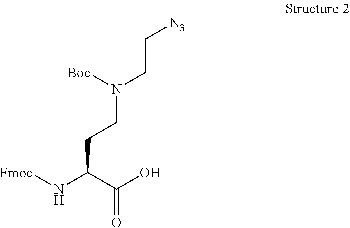

In some implementations, the unnatural lysine residue, or a stereoisomer, tautomer, or pharmaceutically acceptable salt or ester thereof, has structure 19.

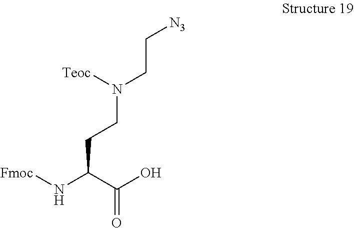

In some implementations, the unnatural lysine residue, or a stereoisomer, tautomer, or pharmaceutically acceptable salt or ester thereof, has structure 1:

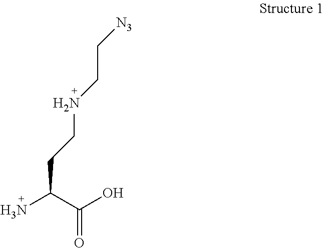 [SMILES: [N-]=[N+]=NCCNCC[C@H](N)C(=O)O]

Also, disclosed herein are implementations of a method for making the unnatural lysine residue, for example, of formula 1 or of Structure 1 or 2. A representative method is provided below by Scheme 1:

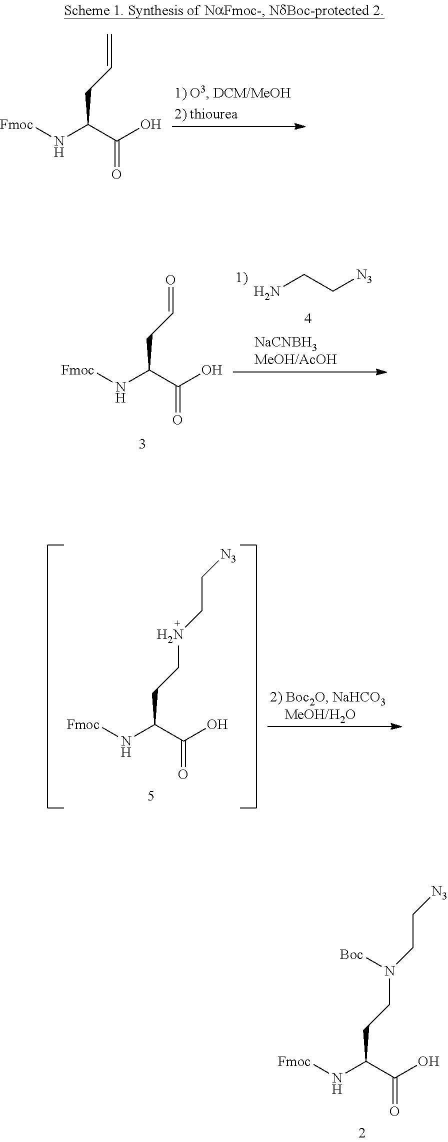

With reference to Scheme 1, an exemplary procedure that can be used in some implementations (reagent amounts, reaction times, and temperatures, etc.) is provided in Example 1. Additional details concerning methods for making a representative unnatural lysine residue as disclosed herein are provided in the Examples section.

In some aspects, a polypeptide is provided, comprising the unnatural lysine provided herein. The polypeptide can be an length. In some examples, the polypeptide is a peptide, such as a β-Hairpin peptide, for example, an amphiphilic cationic β-hairpin peptide. In some aspects, the amphiphilic cationic β-hairpin peptide comprises an amino acid sequence set forth as:

| (SEQ ID NO: 6) | |
|---|---|
| | (XZ)ₙ X^{D}PPX (ZX)ₙ; |
| (SEQ ID NO: 7) | |
| | (XZ)ₙ X^{D}PGX (ZX)ₙ; |
| | or |
| (SEQ ID NO: 8) | |
| | (XZ)ₙ XNGX (ZX)ₙ; |

and
wherein each X is individually selected from V, I, L, M, T, F, W, and Y; at least one Z is the unnatural lysine and the remaining Z are individually selected from any amino acid; X^{D} is a D-amino acid selected from a V, I, L, M, T, F, W, and Y D-amino acid; and n is from 3 to 5 (such as 3, 4, or 5).

In some aspects, the amphiphilic cationic β-hairpin peptide comprises an amino acid sequence set forth as:

| (SEQ ID NO: 6) | |
|---|---|
| | (XZ)ₙ X^{D}PPX (ZX)ₙ; |

wherein each X is individually selected from V, I, L, M, T, F, W, and Y; at least one Z is the unnatural lysine and the remaining Z are individually selected from any amino acid; X^{D} is a D-amino acid selected from a V, I, L, M, T, F, W, and Y D-amino acid; and n is from 3 to 5 (such as 3, 4, or 5).

In some aspects, the amphiphilic cationic β-hairpin peptide comprises or consists of an amino acid sequence set forth as any one of

| (SEQ ID NO: 1) | |
|---|---|
| | VLTZVZTZV^{D}PPTZVQVZVFV; |
| (SEQ ID NO: 2) | |
| | VZTZVEVEV^{D}PPTEVETZVZV; |
| (SEQ ID NO: 3) | |
| | VETZVEVZV^{D}PPTEVZTEVZV; |
| (SEQ ID NO: 4) | |
| | VZVZVZVZV^{D}PPTZVZVZVZV; |
| | and |
| (SEQ ID NO: 5) | |
| | VZVZVZVZV^{D}PPTZVEVZVZV; |

and
wherein each Z is the unnatural lysine.

The peptide folds into a β-hairpin conformation when dissolved at 2.0% w/v in 50 mM Bis Tris Propane, pH 7.4, 150 mM NaCl, at 25° C.

In some aspects, the amphiphilic cationic β-hairpin peptide is acetylated at the N-terminus, amidated at the C-terminus, or acetylated at the N-terminus and amidated at the C-terminus. For example, the amphiphilic cationic β-hairpin peptide in the hydrogel comprises or consists of any one of:

| | XP1 |
|---|---|
| (SEQ ID NO: 1) | |
| | Ac-VLTZVZTZV^{D}PPTZVQVZVFV-NH₂ |
| | XP2 |
| (SEQ ID NO: 2) | |
| | VZTZVEVEV^{D}PPTEVETZVZV-NH₂ |
| | XP3 |
| (SEQ ID NO: 3) | |
| | VETZVEVZV^{D}PPTEVZTEVZV-NH₂ |
| | XP4 |
| (SEQ ID NO: 3) | |
| | VETZVEVZV^{D}PPTEVZTEVZV |
| | XP5 |
| (SEQ ID NO: 2) | |
| | Ac-VZTZVEVEV^{D}PPTEVETZVZV |
| | XP6 |
| (SEQ ID NO: 3) | |
| | Ac-VETZVEVZV^{D}PPTEVZTEVZV |
| | XP7 |
| (SEQ ID NO: 4) | |
| | VZVZVZVZV^{D}PPTZVZVZVZV-NH₂ |
| | XP8 |
| (SEQ ID NO: 5) | |
| | VZVZVZVZV^{D}PPTZVEVZVZV-NH₂ |

wherein each Z is the unnatural lysine, Ac indicates that the peptide is acetylated at the N-terminus, and NH₂ indicates that the peptide is amidated at the C-terminus.

### IV. Peptide Hydrogel and Use Thereof

In several aspects, a peptide hydrogel is provided that formed using an amphiphilic cationic β-Hairpin peptide comprising the novel unnatural lysine described herein.

In some aspects, a peptide hydrogel is provided that is formed from an amphiphilic cationic β-hairpin peptide, wherein the amphiphilic cationic β-hairpin peptide comprises an unnatural lysine according to Formula 2:

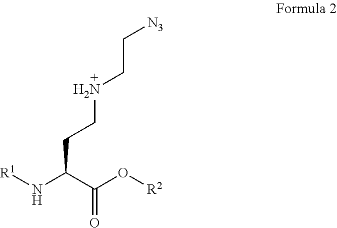

wherein R¹ is another amino acid in the peptide or the N-terminus of the peptide and R² is another amino acid in the peptide or the C-terminus of the peptide.

The peptide hydrogel is formed from a fibrillar network of an amphiphilic cationic peptide that is in a β-hairpin conformation. The β-strand regions of the hairpin contain alternating sequences of hydrophobic (e.g., valine) and hydrophilic (charged) residues (e.g., lysine) such that in the folded state, one face (e.g., the valine-rich face) of the peptide is hydrophobic and the opposing face (e.g., the lysine rich face) is lined with positively charged side chains and is hydrophilic. This amphiphilic arrangement facilitates inter-molecular peptide interactions, and the fibril arrangement necessary for hydrogel formation.

Self-assembly is facilitated facially by hydrophobic association of the hydrophobic faces of folded hairpins and laterally via H-bond formation and hydrophobic van der Waals contacts between neighboring hairpins. Detailed knowledge of these parameters allows control of the self-assembly process and thus the ultimate hydrogel material properties. For example, under folding conditions peptides may adopt a desired secondary structure (e.g., may adopt an amphiphilic β-hairpin structure where one face of each β-strand in the hairpin is lined with hydrophobic residues and the other face is lined with hydrophilic residues). For example, intramolecular folding is dictated by the alleviation of charge density on the hydrophilic face upon folding, the formation of intramolecular hydrophobic van der Waals interactions, the formation of intramolecular hydrogen bonds between β-strands within the hairpin, and the turn propensity of the β-turn sequence included in the peptide.

Thus, peptides for use in the hydrogel disclosed herein can be constructed to have desired characteristics by varying one or more of at least the following parameters: 1) electrostatics, for example, by varying the charge within the peptide intramolecular folding and self-assembly rates can be varied; 2) van der Waals interactions, for example, constructing peptides having varying a) lateral and facial intermolecular hydrophobic interactions and/or b) intramolecular hydrophobic interactions, allowing variation in the folding and self-assembly of the peptides as well as the material properties of the hydrogel; 3) hydrogen bonding, for example peptides may be constructed with varying a) intramolecular and/or b) intermolecular hydrogen bond formation to vary the folding, self-assembly and final material properties; and 4) turn sequence, for example, the turn region of peptides of the invention may be designed to control folding and thus trigger self-assembly.

In several embodiments, the peptide includes high β-sheet propensity residues flanking an intermittent four residue turn sequence. Polar and apolar residues may be arranged sequentially in the strand regions to afford amphiphilic surfaces when the peptide is folded in a β-hairpin conformation. For the four residue turn sequence, the peptide typically includes four residues (termed i, i+1, i+2, and i+3) that form a type II′ β-turn. In the disclosed azido-MAX8 peptide, these four residues are V^{D}PPT, and the type II′ β-turn is defined by the dihedral angles (Phi and Psi) adopted by the ^{D}pp portion of the turn sequence, where ‘^{D}’ denotes D-stereochemistry of the first proline residue. The preferred phi and psi dihedral angles (degrees) that define a type II′ turn are: residue i+1 (60, −120); residue i+2 (−80,0). However, these values can vary by +/−20 degrees and the peptide can still form the appropriate β-turn structure.

In one particular embodiment, azido-MAX8, a 20-residue peptide is composed of high (β-sheet propensity valine and lysine residues flanking an intermittent tetrapeptide—V^{D}PPT—designed to adopt type-II′ β-turn structure. In addition to incorporating local design elements to stabilize hairpin structure, nonlocal effects were also considered by arranging the polar and apolar residues flanking the β-turn in an alternating fashion to favor β-hairpin formation in the self-assembled state. In addition, a β-branched residue was placed at the i-position of the turn to enforce a trans prolyl amide bond geometry at the i+1 position. This design element ensures that under folding conditions, intramolecular folding of monomeric hairpins is favored prior to self-assembly. A cis prolyl bond, which is designed against, could result in the presentation of individual β-strands within each monomer in an extended conformation. Peptides capable of adopting both cis and trans conformers could undergo indiscriminant self-association of extended and correctly folded monomers and may be actively designed against.

In some aspects, the amphiphilic cationic β-hairpin peptide in the hydrogel comprises an amino acid sequence set forth as:

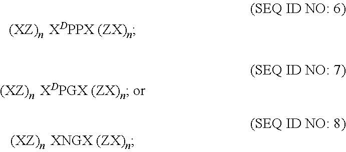

and
wherein each X is individually selected from V, I, L, M, T, F, W, and Y; at least one Z is the unnatural lysine and the remaining Z are individually selected from any amino acid; X^{D} is a D-amino acid selected from a V, I, L, M, T, F, W, and Y D-amino acid; and n is from 3 to 5 (such as 3, 4, or 5).

In some aspects, the amphiphilic cationic β-hairpin peptide in the hydrogel comprises an amino acid sequence set forth as:

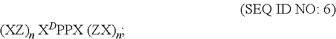

wherein each X is individually selected from V, I, L, M, T, F, W, and Y; at least one Z is the unnatural lysine and the remaining Z are individually selected from any amino acid; X^{D} is a D-amino acid selected from a V, I, L, M, T, F, W, and Y D-amino acid; and n is from 3 to 5 (such as 3, 4, or 5).

In some aspects, the amphiphilic cationic β-hairpin peptide in the hydrogel comprises or consists of an amino acid sequence set forth as any one of

| (SEQ ID NO: 1) | |
|---|---|
| | VLTZVZTZV^{D}PPTZVQVZVFV; |
| (SEQ ID NO: 2) | |
| | VZTZVEVEV^{D}PPTEVETZVZV; |
| (SEQ ID NO: 3) | |
| | VETZVEVZV^{D}PPTEVZTEVZV; |
| (SEQ ID NO: 4) | |
| | VZVZVZVZV^{D}PPTZVZVZVZV; |
| | and |
| (SEQ ID NO: 5) | |
| | VZVZVZVZV^{D}PPTZVEVZVZV; |

and
wherein each Z is the unnatural lysine.

The peptide folds into a β-hairpin conformation when dissolved at 2.0% w/v in 50 mM Bis Tris Propane, pH 7.4, 150 mM NaCl, at 25° C.

In some aspects, the amphiphilic cationic β-hairpin peptide in the hydrogel is acetylated at the N-terminus, amidated at the C-terminus, or acetylated at the N-terminus and amidated at the C-terminus. For example, the amphiphilic cationic β-hairpin peptide in the hydrogel comprises or consists of any one of:

| | XP1 |
|---|---|
| (SEQ ID NO: 1) | |
| | Ac-VLTZVZTZV^{D}PPTZVQVZVFV-NH₂ |
| | XP2 |
| (SEQ ID NO: 2) | |
| | VZTZVEVEV^{D}PPTEVETZVZV-NH₂ |
| | XP3 |
| (SEQ ID NO: 3) | |
| | VETZVEVZV^{D}PPTEVZTEVZV-NH₂ |
| | XP4 |
| (SEQ ID NO: 3) | |
| | VETZVEVZV^{D}PPTEVZTEVZV |
| | XP5 |
| (SEQ ID NO: 2) | |
| | Ac-VZTZVEVEV^{D}PPTEVETZVZV |
| | XP6 |
| (SEQ ID NO: 3) | |
| | Ac-VETZVEVZV^{D}PPTEVZTEVZV |
| | XP7 |
| (SEQ ID NO: 4) | |
| | VZVZVZVZV^{D}PPTZVZVZVZV-NH₂ |
| | XP8 |
| (SEQ ID NO: 5) | |
| | VZVZVZVZV^{D}PPTZVEVZVZV-NH₂ |

wherein each Z is the unnatural lysine, Ac indicates that the peptide is acetylated at the N-terminus, and NH₂ indicates that the peptide is amidated at the C-terminus.

The amphiphilic cationic peptide can fold into an β-hairpin conformation comprising a β-turn, two β-strands, a hydrophobic face, and a hydrophilic face under appropriate conditions (e.g., 2.0% w/v peptide in 50 mM Bis Tris Propane, pH 7.4, 150 mM NaCl, at 25° C.). Under the appropriate conditions, the amphiphilic cationic peptide self-assembles into a fibrillar network wherein the peptide is folded in a β-hairpin conformation in the fibrillar state.

The peptide hydrogel can readily be made by preparing an aqueous solution comprising one or more of the cationic amphiphilic peptides (such as azido-MAX8) as disclosed herein and altering one or more characteristics of the solution, wherein a hydrogel is formed. The characteristic altered may be any characteristic that results in formation of a hydrogel upon its alteration. Suitable examples include, but are not limited to, ionic strength, temperature, concentration of a specific ion, and pH. In particular embodiments, the character altered may be the pH of the solution. The cationic amphiphilic peptide forms a hydrogel at a pH of about 7 or higher. Increasing pH and increasing ionic strength both encourage hydrogel formation, and the two effects are roughly additive. Thus, the lower the pH, the higher the salt concentration necessary for hydrogel formation. In some embodiments, the hydrogel can be formed in a container (such as a syringe), for example a closed container.

In some embodiments, altering one or more characteristics of the solution results in a salt concentration of from about 10 mM to about 400 mM, such as about 50 to about 300 mM, about 100 to about 200 mM, or about 150 mM. Any salt may be used, for example, KCl, NaCl, MgCl₂, KF, MgSO₄, etc. In one embodiment, the salt may be NaCl. In some embodiments, the solution may have a desired pH, for example, a pH of from about 7 to about 9, a pH of from about 7.5 to about 8.5, a pH of from about 7.0 to about 8.0, or a pH of about 7.4, which may stay the same or be changed upon formation of the hydrogel.

In one non-limiting example, the hydrogel is formed in 50 mM Bis Tris Propane (BTP), 150 mM NaCl, pH 7.4. Any buffer system can be used except phosphate based buffer systems, as phosphate buffers are known to precipitate β-hairpin peptides. Accordingly, peptide hydrogels including a cationic amphiphilic peptide can simply be formed by, for example, adding buffer of appropriate ionic strength to an aqueous solution of unfolded peptide; drawing the resulting solution into a syringe; and allowing it to gel at 25° C. directly in the syringe.

The peptide hydrogel is a well hydrated solid material and has a stiffness greater than 40 Pascal (Pa), as measured by the storage modulus G′ at a strain of 0.2%. Above approximately 40 Pa the material is a self-supporting solid gel material. The stiffness can reach greater than 10,000 Pa at higher peptide concentration. The hydrogel typically contains at least 0.5 wt % of the amphiphilic cationic peptide in an aqueous medium. For example, the disclosed hydrogel may have varying amounts of the amphiphilic cationic peptide material. For example, the hydrogel may be formed comprising a percent by weight of the amphiphilic cationic peptide of from about 0.25% w/v to about 4.0% w/v, from about 0.25% w/v to about 3.0% w/v, from about 0.25% w/v to about 2.0% w/v, from about 0.25% w/v to about 1.0% w/v, from about 0.5% w/v to about 4.0% w/v, from about 0.5% w/v to about 3.0% w/v, from about 0.5% w/v to about 2.0% w/v, from about 0.5% w/v to about 1.0% w/v, from about 1.0% w/v to about 4.0% w/v, from about 1.0% w/v to about 3.0% w/v, from about 1.0% w/v to about 2.0% w/v, from about 2.0% w/v to about 4.0% w/v, or from about 2.0% w/v to about 3.0% w/v.

In one aspect, the amount by weight of the amphiphilic cationic peptide and the kinetics of gelation may be varied to produce a hydrogel having a desired modulus (stiffness). Hydrogels of the invention may have a modulus from about 40 Pascal (Pa) to about 50,000 Pa, from about 40 Pa to about 25,000 Pa, from about 40 Pa to about 10,000 Pa, from about 40 Pa to about 5,000 Pa, from about 40 Pa to about 1,000 Pa, from about 40 Pa to about 500 Pa, from about 40 Pa to about 100 Pa, from about 100 Pa to about 50,000 Pa, from about 100 Pa to about 25,000 Pa, from about 100 Pa to about 10,000 Pa, from about 100 Pa to about 5,000 Pa, from about 100 Pa to about 2,000 Pa, from about 100 Pa to about 1,000 Pa, from about 100 Pa to about 500 Pa, or from about 100 Pa to about 250 Pa.

The resultant hydrogel is mechanically rigid and displays shear-thinning/recovery behavior. This characteristic provides a free flowing suspension during the application of shear and complete reformation of the gel network (self-healing) after cessation of the shear. This combination of shear thinning and self-healing allows material formation in a spatially resolved manner. For example, in some embodiments, one of ordinary skill in the art can inject or spray (shear thin) a pre-formed hydrogel into a target location in a subject where it self-heals and reforms the hydrogel. The shear stress converts the gel to a lower viscosity, flowable fluid. The shear stress is relieved when the fluid exits the syringe or spray nozzle and the gel quickly self-heals, recovering its original mechanical rigidity. This shear-thinning/recovery mechanism allows the nanoparticle-hydrogel composite to be easily delivered by syringe or spray to the target location in the subject.

The amphiphilic cationic peptides for use in the disclosed embodiments can be peptides from about 20 to about 75 residues (e.g., from about 20 to about 50 residues, from about 20 to about 40 residues, from about 20 to about 30 residues, or from about 20 to about 25 residues, (“about” refers to plus or minus 2 residues). In some embodiments, the peptides for use in the disclosed embodiments can be from 20 to 75 residues (e.g., from 20 to 50 residues, from 20 to 40 residues, from 20 to 30 residues, or from 20 to 25 residues). In some embodiments, the peptide can be no more than 50 residues, such as no more than 30 residues or no more than 20 residues. In additional embodiments, the peptide can be 20, 25, 30, 35, 40, 45, or 50, residues in length. In some embodiments, the peptide can be 20 amino acids in length.

The amphiphilic cationic peptides for use in the disclosed embodiments can be synthesized using any appropriate technique, such as automated solid phase procedures. The amphiphilic cationic peptides may incorporate one or more modified amino acid residues (e.g., D-amino acids, homologs of naturally occurring amino acids, amino acids with modified side chains, etc.). Exemplary techniques and procedures for solid phase synthesis are described in Solid Phase Peptide Synthesis: A Practical Approach, by E. Atherton and R. C. Sheppard, published by IRL, Oxford University Press, 1989. Alternatively, peptides may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). Other methods useful for synthesizing the peptides of the disclosure are described in Nakagawa et al., J. Am. Chem. Soc. 107:7087-7092, 1985.

Additional exemplary techniques for peptide synthesis are taught by Bodanszky, M. and Bodanszky, A., The Practice of Peptide Synthesis, Springer Verlag, New York, 1994; and by Jones, J., Amino Acid and Peptide Synthesis, 2nd ed., Oxford University Press, 2002. The Bodanszky and Jones references detail the parameters and techniques for activating and coupling amino acids and amino acid derivatives. Moreover, the references teach how to select, use and remove various useful functional and protecting groups. Peptides of the disclosure can also be readily purchased from commercial suppliers of synthetic peptides once the supplier is provided with the sequence of the peptide. Such suppliers include, for example, Advanced ChemTech (Louisville, KY), Applied Biosystems (Foster City, CA), Anaspec (San Jose, CA), and Cell Essentials (Boston, MA).

Following synthesis, exemplary techniques for peptide purification include reverse phase chromatography, high performance liquid chromatography, ion exchange chromatography, size exclusion chromatography, affinity chromatography, and gel electrophoresis. The actual conditions used to purify a particular peptide, or a modified form thereof, will depend, in part, on synthesis strategy and on factors such as net charge, hydrophobicity, hydrophilicity, and the like.

The amphiphilic peptides disclosed herein are cationic. Accordingly, in typical embodiments involving a crystallized small molecule dispersed in the peptide hydrogel, the small molecule has a neutral or net positive charge to prevent binding of the drug to the hydrogel matrix. Depending on the drug, the neutral or net positive charge may lead to varying retention time in the peptide hydrogel.

In some aspects, the peptide hydrogel is labeled with a detectable marker. The azide group of the unnatural lysine in the peptide hydrogel is readily reacted with a SPAAC probe (e.g., BCN or DBCO) linked to the detectable marker. In some aspects, one or more of the unnatural lysine residues in the amphiphilic cationic β-hairpin peptide of the peptide hydrogel are linked to a detectable marker by Strain Promoted Azide-Alkyne Cycloaddition (SPAAC) chemistry via the terminal azide group of the unnatural lysine. In some aspects, the detectable marker comprises a contrast agent (e.g., containing a triiodo group) for contrast enhanced computed tomography (CECT) imaging. In some aspects, the detectable marker comprises a contrast agent for CECT imaging, such as any one of Triiodobenzoic acid (TIBA), iohexol, iopromide, iothalamate, ioxaglate, and iodixanol. In some aspects, the detectable marker comprises a contrast agent for CECT imaging, such as any one of:

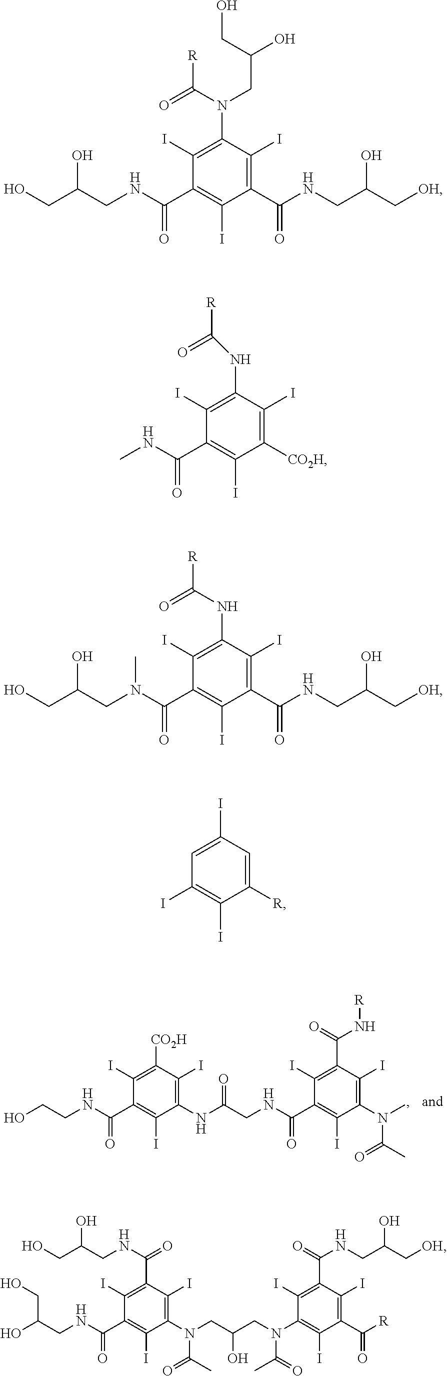

wherein R is a linkage to the unnatural lysine residues in the amphiphilic cationic β-hairpin peptide by SPAAC chemistry.

Labeling of the peptide hydrogel with the detectable marker can be accomplished using any suitable method. In several aspects, the peptide hydrogel is labeled with the detectable marker by SPAAC ligation via the terminal azide group of the unnatural lysine in the amphiphilic cationic β-hairpin peptide of the peptide hydrogel. In such aspects, the detectable maker (e.g., triiodo group) is linked to a SPAAC probe, such as BCN or DBCO, and incubated with the peptide hydrogel under conditions suitable for SPAAC ligation.

The peptide hydrogel and triiodo-labeled peptide hydrogel provided herein displays shear-thin/recovery mechanical properties, which allow the triiodo-labeled peptide hydrogel to be delivered locally to a body cavity, for example, via percutaneous or surgical access by syringe injection.

Also provided is a method of contrast enhanced computed tomography (CECT) imaging of a subject, comprising administering an effective amount of the triiodo-labeled peptide hydrogel provided herein to a target location in the subject, and conducting a contrast CT scan of the target location. An effective amount of the triiodo-labeled peptide hydrogel is an amount that is detectable with suitable specificity and sensitivity by CT imaging. In several aspects, the triiodo-labeled peptide hydrogel is administered to the target location in the subject by syringe injection.

One skilled in the art can readily determine an effective amount of a disclosed triiodo-labeled peptide hydrogel to be administered to a target location in a subject, for example, taking into account factors such as the target location, the age, health and sex of the subject, the size (e.g., weight and/or height) of the subject, and the specific route of administration. The particular mode of administration and the dosage regimen will be selected by the attending clinician, taking into account the particulars of the case (e.g. the subject, the disease, the disease state involved, etc.). The triiodo-labeled peptide hydrogel can be formulated in unit dosage form, suitable for individual administration of precise dosages.

### EXAMPLES

The following examples are provided to illustrate particular features of certain embodiments, but the scope of the claims should not be limited to those features exemplified.

### Example 1

Preparation of a Novel Unnatural Lysine for SPAAC Reactions This example describes a new positively charged azido-amino acid for Strain-Promoted Azide Alkyne Cycloaddition (SPAAC) applications that overcomes possible solubility limitations of commonly used azido lysine, especially in systems with numerous ligation sites. The residue is easily synthesized, compatible with Fmoc-based solid-phase peptide synthesis employing a range of coupling conditions, and offers efficient second-order rate constants in SPAAC ligations employing DBCO (0.34 M⁻¹ s⁻¹) and BCN (0.28 M⁻¹s⁻¹).

Strain-Promoted Azide Alkyne Cycloaddition has become a widely used tool for the selective modifications of biomolecules in vitro and in vivo for numerous chemical biology and materials applications. This reaction is advantageous because of its chemoselectivity, reaction rates, and compatibility with aqueous physiological environments. However, the modification of peptides, proteins, and other macromolecules with azido or strained alkyne groups for SPAAC reactions can affect the macromolecule's physical, structural, and functional properties, particularly when residues in the native primary sequence are altered or when multiple modifications are required. For example, incorporating ligation handles can dramatically increase macromolecular hydrophobicity affecting solubility and aggregation propensity. In some cases, organic solvents are needed to aid solubility, but at the risk of influencing macromolecular structure.

A case in point is azido lysine (^{N3}K), which is commonly used to introduce an azide into peptides and proteins by solid-phase peptide synthesis (SPPS), often replacing a Lys residue in the native primary sequence. However, this replacement eliminates the Lys side-chain amine group that provides positive charge, which in turn, facilitates solubility. Further, the side-chain positive charge may also be required for molecular interactions relevant to structure and function. In some examples, a problematic decrease in the solubility of peptides when ^{N3}K was incorporated at the expense of Lys was observed.

In this example, we designed the positively charged azido-containing amino acid 1:

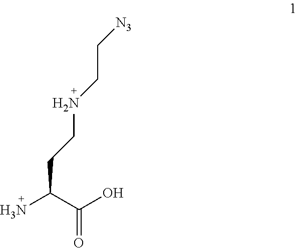 [SMILES: [N-]=[N+]=NCCNCC[C@H](N)C(=O)O]

Amino acid 1 addresses the issue of decreased macromolecule solubility that often accompanies replacing Lys with ^{N3}K. Residue 1 contains the required azido group but also a secondary amine in its side-chain, which is protonated at physiological pH, greatly aiding solubility. The synthesis of the NαFmoc-, NδBoc-protected analog (2) of residue 1, shown in Scheme 1, is simple, high yielding, scalable, and uses inexpensive starting materials. Protected 2 is compatible with standard Fmoc SPPS and numerous copies of the residue can be incorporated into a single peptide while minimally affecting solubility. Importantly, this new side-chain offers similar reaction kinetics with strained alkynes as compared to other azides.

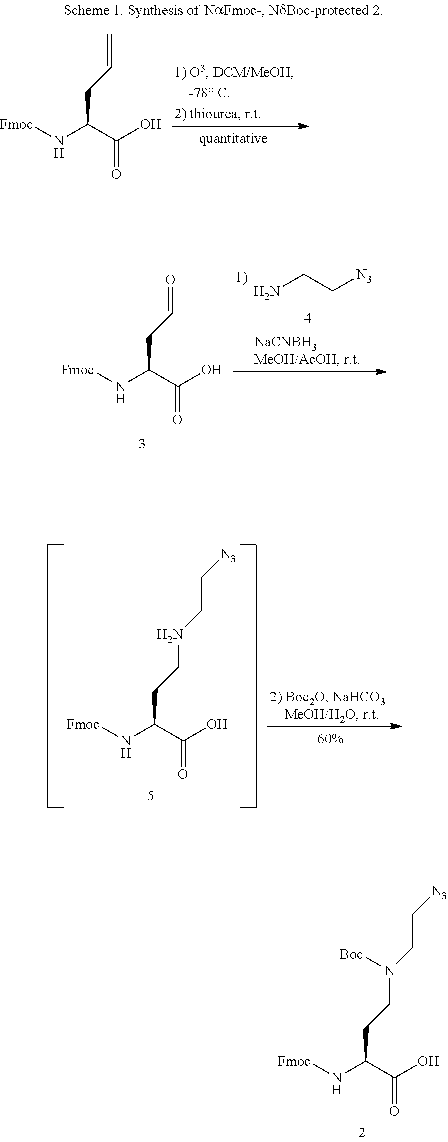

With respect to synthesis, several approaches have been reported for azido-containing amino acids. For example, Hoffmann rearrangement of Fmoc-protected Gln or Asn followed by diazo transfer affords the corresponding azido side-chain. (Lau and Spring, Efficient Synthesis of Fmoc-Protected Azido Amino Acids. Synlett 2011, 2011 (13), 1917-1919; Pïcha et al., Optimized syntheses of Fmoc azido amino acids for the preparation of azidopeptides. J. Pep. Sci. 2017, 23 (3), 202-214.

Another common strategy involves the mesylation or bromination of an hydroxyl side-chain and subsequent substitution by sodium azide. (Schmidt et al., Synthesis of Enantiomerically Pure and Compatibly Protected (2S,3R)- and (2S,3S)-Diaminobutyric Acids. Synth. 1992 (12), 1201-1202; Roth and Thomas, A Concise Route to 1-Azidoamino Acids: 1-Azidoalanine, 1-Azidohomo-alanine and 1-Azidonorvaline. Synlett. 2010 (04), 607-609.) Although useful, this approach requires Bocto-Fmoc protecting group swapping. Further, the multi-step preparation of residues containing aromatic azides and branched azido side-chains have been reported. (Tookmanian, Fenlon, and Brewer, Synthesis and protein incorporation of azido-modified unnatural amino acids. RSC Adv. 2015, 5 (2), 1274-1281; Richardson et al., Synthesis and Explosion Hazards of 4-Azido-1-phenylalanine. J. Org. Chem. 2018, 83 (8), 4525-4536; Pitteloud, Bionda, and Cudic, Direct access to side chain N,N′-diaminoalkylated derivatives of basic amino acids suitable for solid-phase peptide synthesis. Amino Acids 2013, 44 (2), 321-333.)

This example illustrates a simple two-step synthesis of 2 starting from commercially available Fmoc-allyl-Gly-OH, Scheme 1. Ozonolysis of the allyl group quantitatively yields aldehyde 3 after reductive work-up using thiourea. Reductive amination of 3 employing 2-azidoethan-1-amine (4) yields azidoamine 5 which is directly Boc-protected in-situ affording NαFmoc-, NδBoc-protected 2. The overall synthesis can be performed at multi-gram scale with only one flash chromatography purification affording 2 in 60% overall yield.

Residue 2 is compatible with both conventional manual- and microwave-assisted Fmoc-based SPPS. We first investigated its coupling efficiency via the preparation of a family of tri-, tetra-, and octapeptides where residue 2 was coupled to the bulky β-branched residues Ile and Val (peptides 6, 8, 9, 10 and 11), small aliphatic Ala (peptide 12), and to the secondary amine of Pro (peptide 7), Scheme 2. We also coupled bulky Trt-protected His and Gln, Pbf-protected Arg, and Boc-protected Ser to residue 2 in a growing chain (peptides 8-11). The purity of all the peptides (6-12) after TFA cleavage and standard work-up ranged from 80-91% as determined by HPLC indicating that residue 2 allows efficient coupling. Next, we evaluated residue 2's compatibility towards four widely used coupling conditions in SPPS, including HATU-DIPEA, DIC-HOAt, PyBOPDIPEA, and microwave-assisted Oxyma-DIC, via the synthesis of tetrapeptide 9. The different coupling conditions resulted in similar peptide purities, 91%, 81%, 87% and 82%, respectively, and indicate broad tolerance to different coupling conditions.

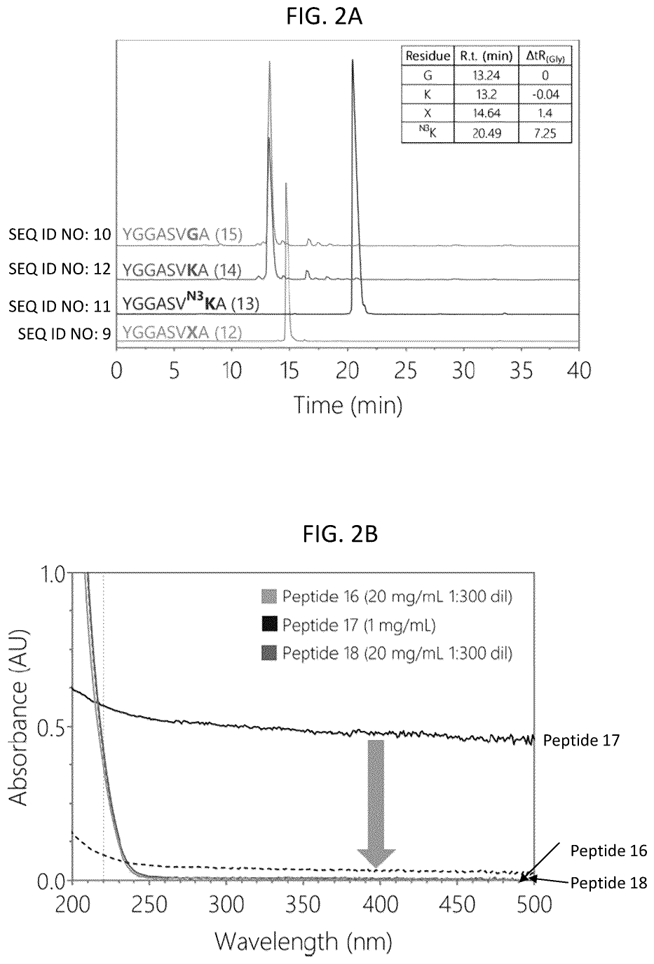

We then determined the intrinsic hydrophilicity/hydrophobicity coefficients (Δt_{R(Gly)}) for residue 1, ^{N3}K, and Lys according to the procedure of Hodges et al. to gain comparative insight into the hydrophilic nature of the new residue. (Kovacs, Mant, and Hodges, Determination of intrinsic hydrophilicity/hydrophobicity of amino acid side chains in peptides in the absence of nearest-neighbor or conformational effects. Peptide Sci. 2006, 84 (3), 283-297.) This method compares RP-HPLC retention times of a peptide containing guest residues at a single position within a sequence to a Gly containing standard. Amino acids with side-chains more hydrophobic than Gly have positive Δt_{R(Gly)} values, and vice-versa. FIG. 2A shows chromatograms of peptides 12, 13, 14 (the Lys control) and 15 (the Gly standard) from which the coefficients are derived. Δt_{R(Gly)} for residue 1 (1.4) is substantially smaller in value than ^{N3}K (7.25) and closer to Gly and Lys (−0.04). This indicates that residue 1 is significantly more hydrophilic than ^{N3}K, and only slightly more hydrophobic than Lys.

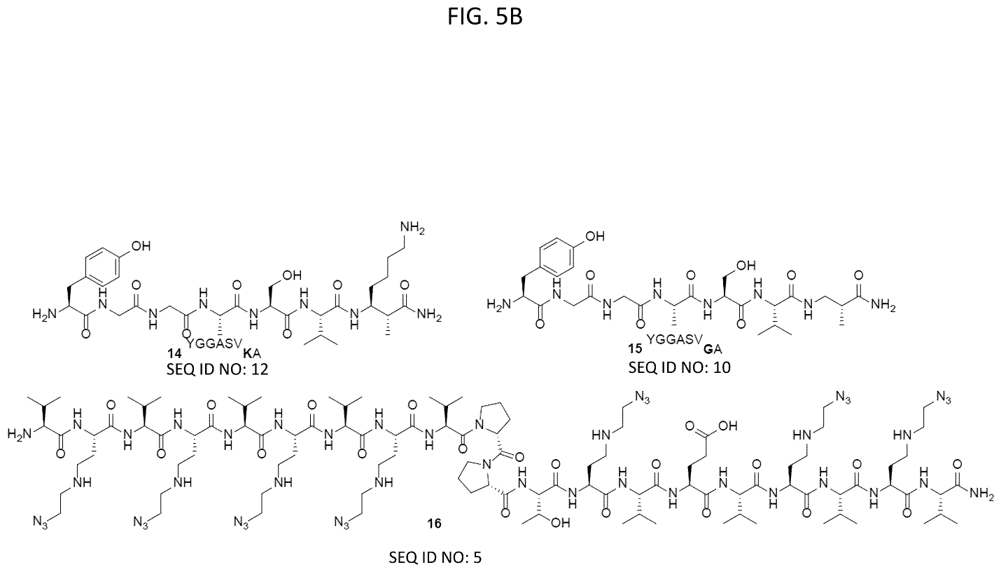

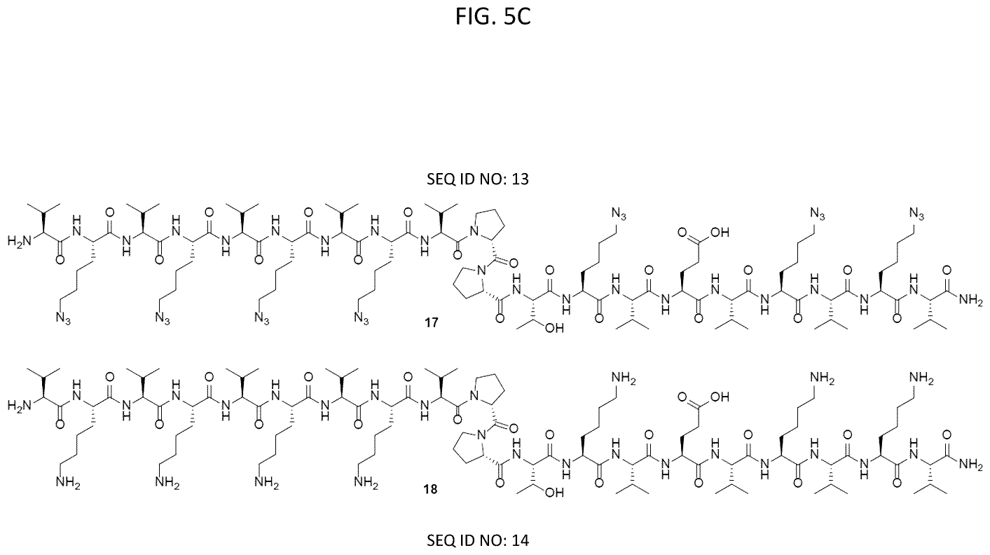

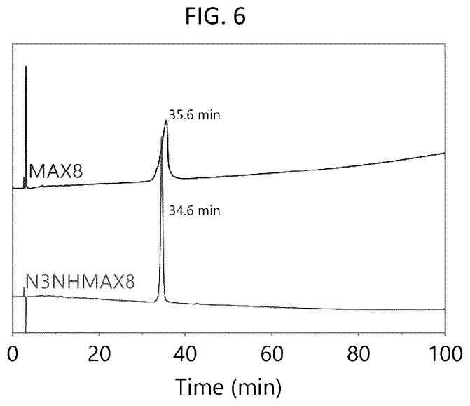

Residue 1's ability to enhance solubility was tested via the amphiphilic peptide 16, which contains 7 copies of 1 along with 9 hydrophobic Val residues. Here, we directly measured solubility via UV-vis spectroscopy of aqueous preparations of peptides before and after centrifugation (FIG. 2B). Peptide 16 (FIG. 5) was fully soluble in water at 20 mg/mL, the highest concentration tested, as was its Lys-containing control peptide 18. (Haines-Butterick et al., Controlling hydrogelation kinetics by peptide design for three-dimensional encapsulation and injectable delivery of cells. Proc. Natl. Acad. Sci. USA, 2007, 104 (19), 7791-7796.) Each peptide showed similar absorbance values at 220 nm independent of centrifugation status and no light scattering at longer wavelengths (FIG. 2B). Further, peptides 16 and 18 had nearly identical RP-HPLC chromatograms (FIG. 6) indicating similar hydrophilic character. In contrast, the ^{N3}K-containing peptide 17 was completely insoluble in water at only 1 mg/mL, showing extensive light scattering before centrifugation and no scattering after, due to sedimentation of aggregates. Taken together, the data show that residue 1 significantly enhances solubility.

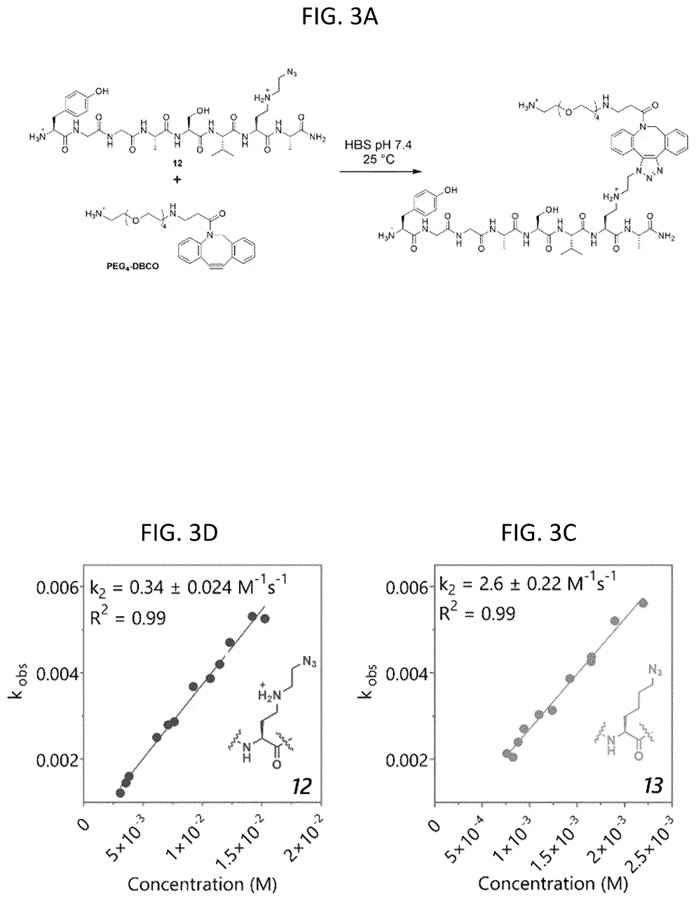

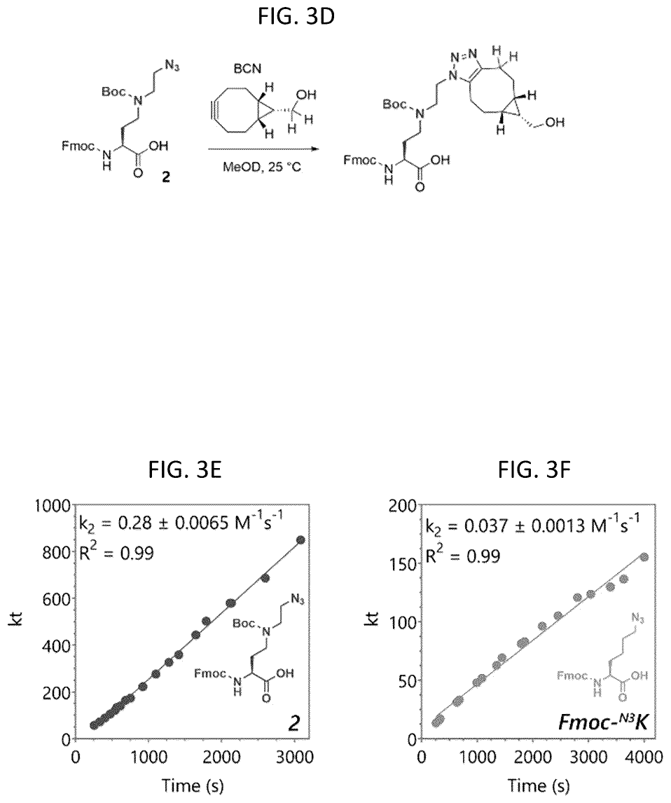

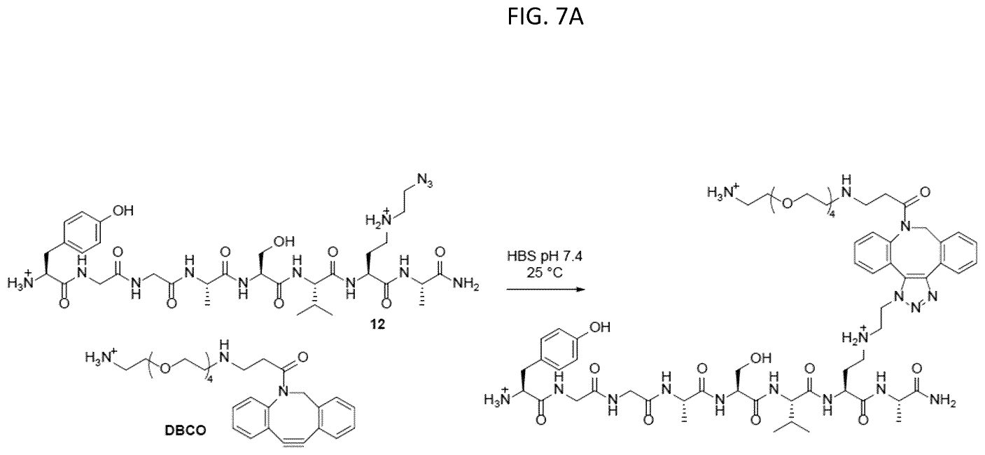

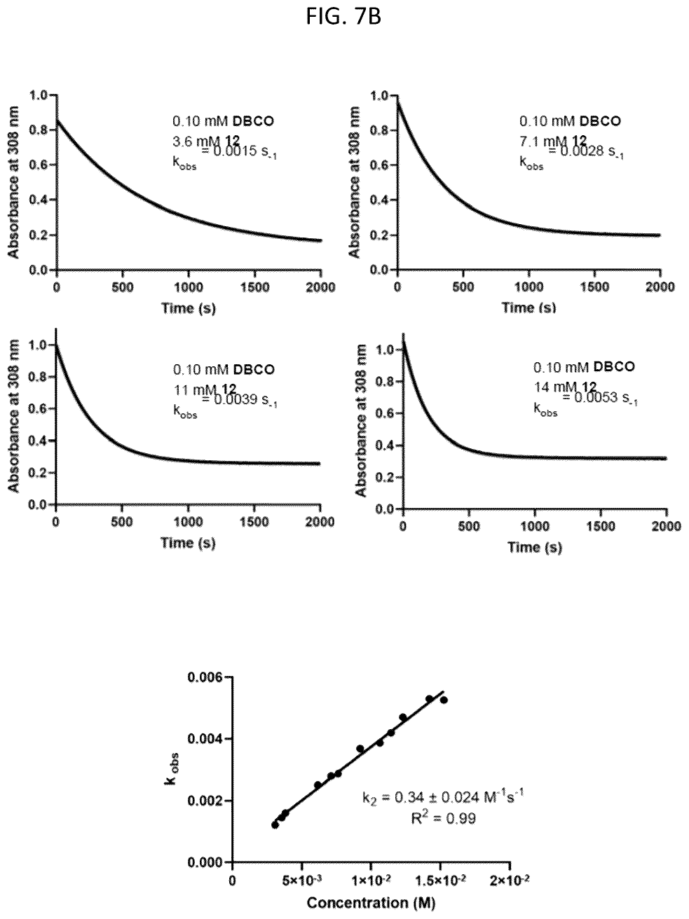

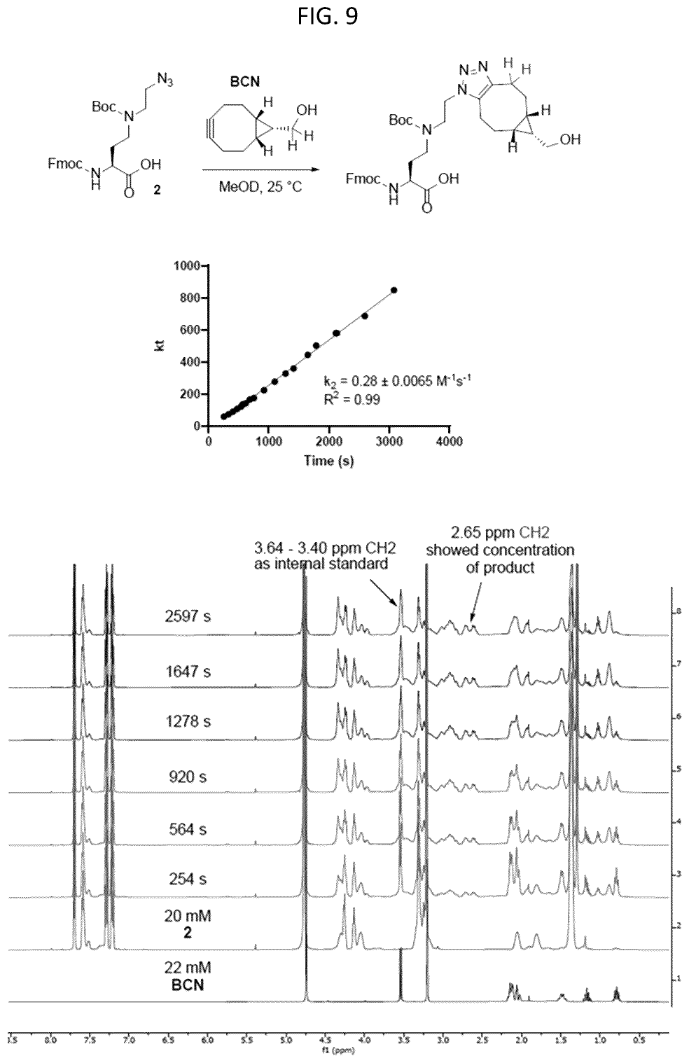

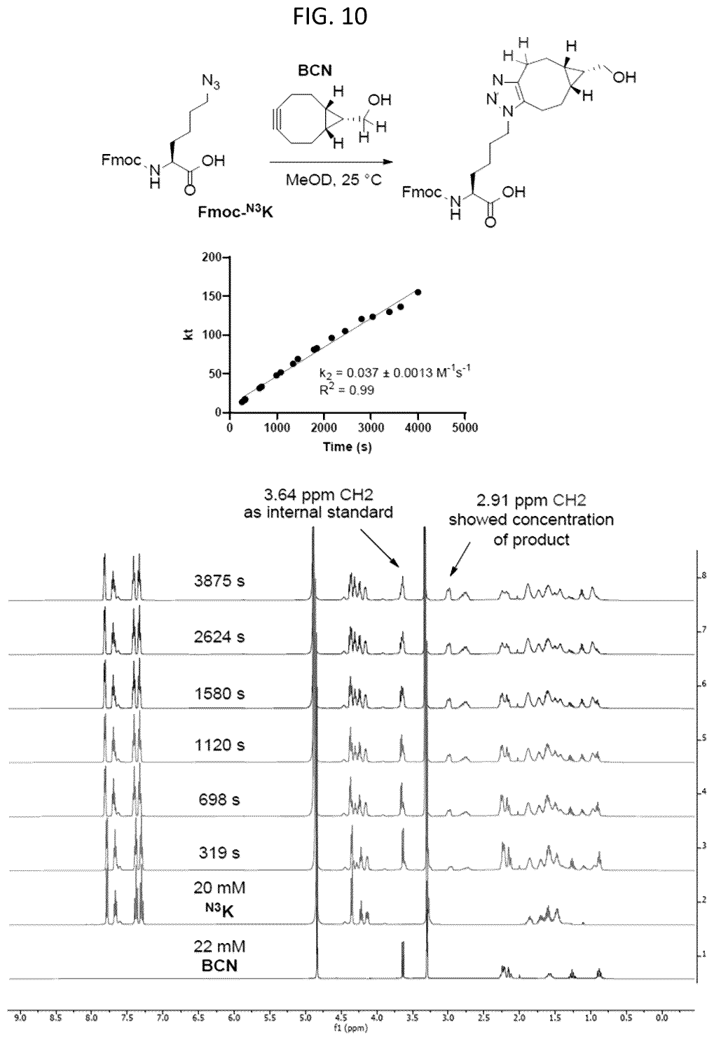

The reaction kinetics of amino acid 1 in SPAAC with two strained cyclooctynes, DBCO and BCN, were determined in comparison with ^{N3}K. First, peptides 12 and 13, which contain residue 1 and ^{N3}K respectively, were independently ligated to PEG-functionalized DBCO. Rates were determined under pseudo-first order conditions by UV-vis spectroscopy at 308 nm (FIGS. 3A-3C, 7, 8). The calculated second order rate constant for peptide 12 was 0.34 M⁻¹ s⁻¹ in HBS buffer, pH 7.4 at 25° C., which is similar to previously reported rate constants for other azides. The ligation rate of peptide 12 is slightly slower than peptide 13 (2.6 M⁻¹s⁻¹), which may be due to hydrophobic interactions between the ^{N3}K side-chain and the aromatic DBCO, facilitating ligation. However, the reaction is fast and completed in about 15 min with concentrations of 0.1 mM DBCO and 14 mM peptide 12 (FIG. 7). We then determined the reaction rate of Boc-protected amino acid 2 itself with BCN in methanol at 25° C. (FIGS. 3D-3E, 9) under second order conditions using NMR and equation 1, resulting in a k₂ of 0.28 M⁻¹s⁻¹, which is comparable with prior Boc-protected amino acids and slightly faster than Fmoc-^{N3}K (0.037 M⁻¹s⁻¹), FIGS. 3F and 10. (Dommerholt et al., Strain-Promoted 1,3-Dipolar Cycloaddition of Cycloalkynes and Organic Azides. Top. Curr. Chem. 2016, 374 (2), 16.) Again, the faster rate of residue 2 may be due to its hydrophobic Boc group promoting BCN association. Taken together, these experiments demonstrate that amino acid 1 is a useful residue for the modification of peptides via SPAAC reactions in water or organic media.

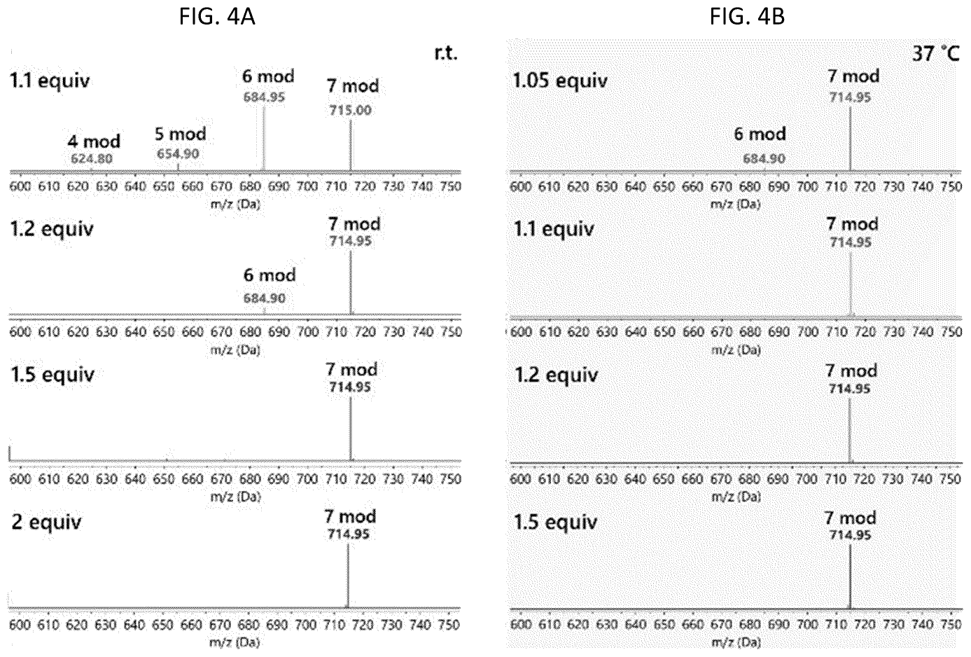

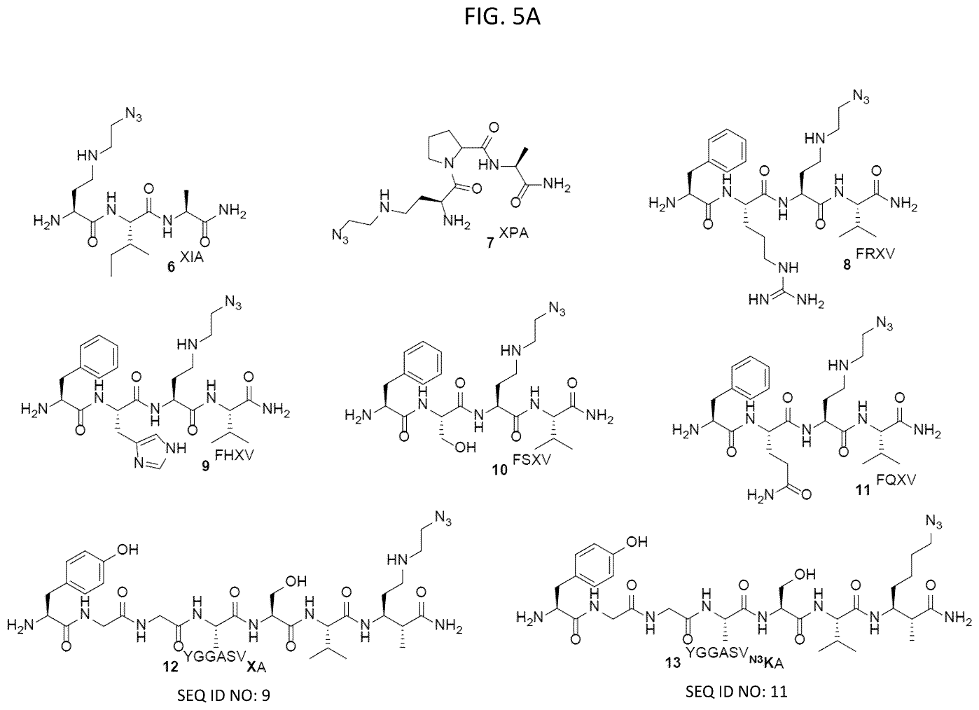

One of the main advantages of residue 1 is its increased hydrophilicity compared to ^{N3}K and ability to impart solubility, which should prove useful in the preparation of highly functionalized peptides and other biomolecules containing multiple ligation sites. To test this assertion, we evaluated the reaction of peptide 16, containing seven copies of residue 1, with BCN. Although the peptide is soluble in water, 10% DMSO is added to solubilize the BCN. Ligation efficiency is dependent on BCN concentration with 1.5 equivalents needed to drive the reaction to completion at room temperature (FIG. 4A). However, when the reaction is performed at 370 C, 1.1 equivalents of BCN is sufficient to obtain the hepta-modified peptide product (FIG. 4B).

In summary, this example illustrated a new unnatural amino acid that contains both a secondary amine and azide in its side chain to impart charge and solubility while allowing efficient SPAAC ligation. The synthesis of 2 is efficient and scalable, resulting in an amino acid compatible with Fmoc-SPPS. Numerous azide residues 1, capable of efficient ligation, can be incorporated into a single peptide without sacrificing solubility nor affecting the peptide's primary charge state. This new amino represents a new tool in the SPAAC toolbox to facilitate macromolecular ligation where solubility and maintenance of primary charge are of concern, especially in highly decorated systems.

### Methods and Materials

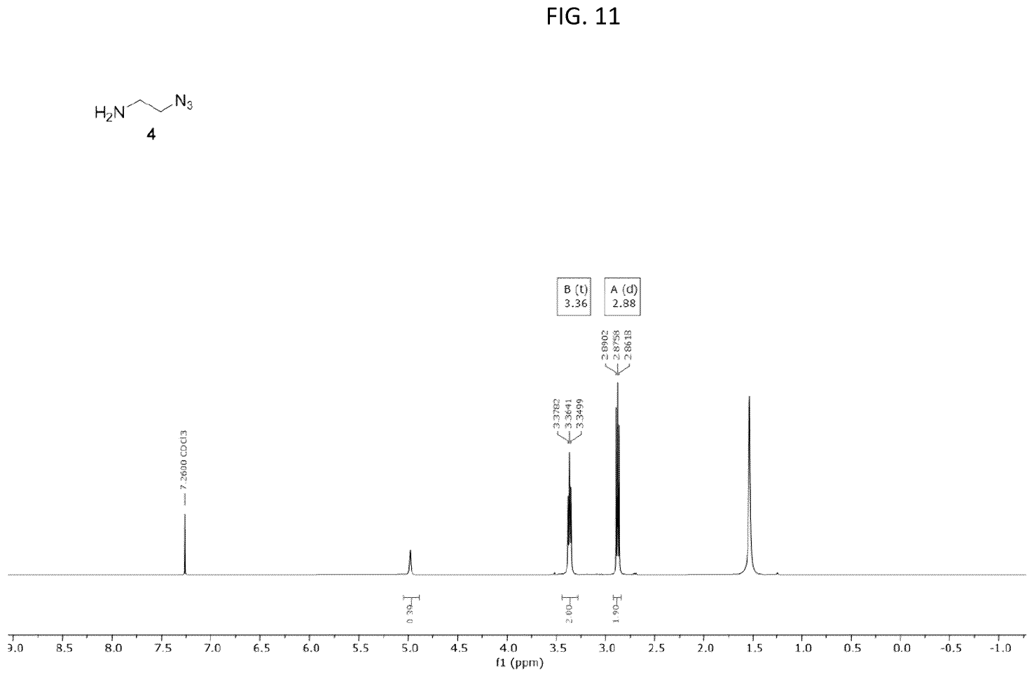

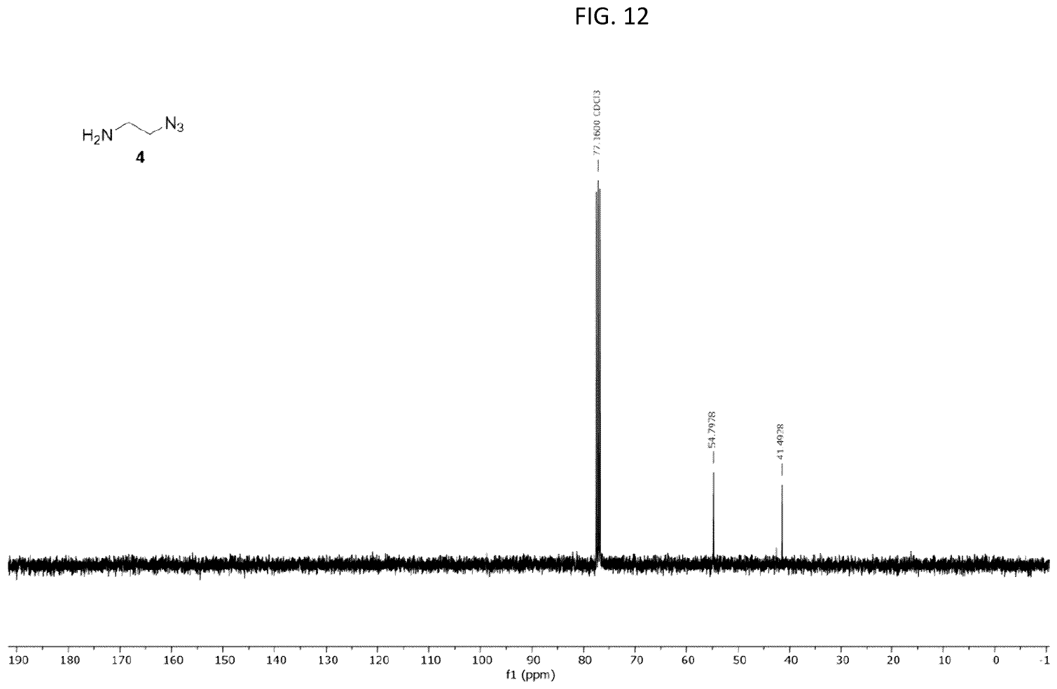

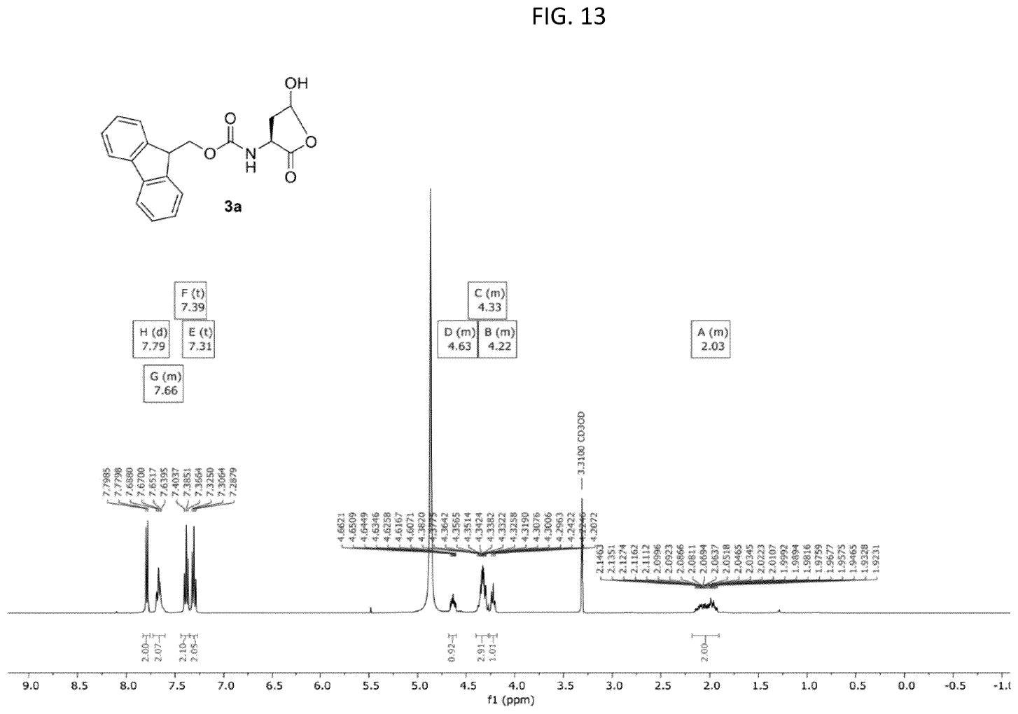

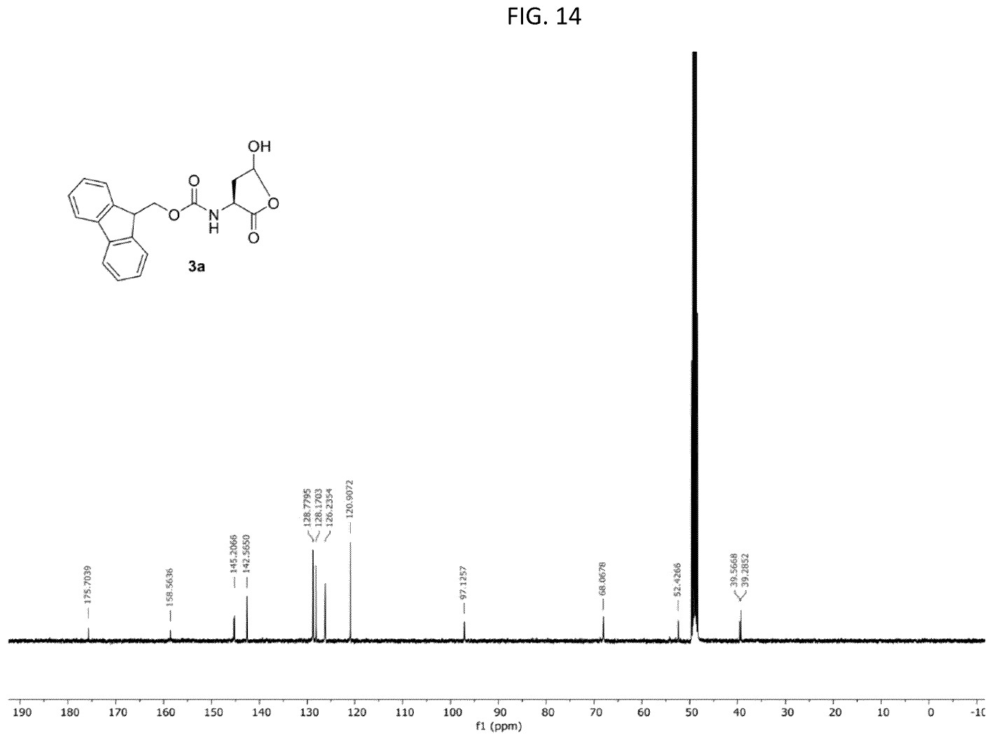

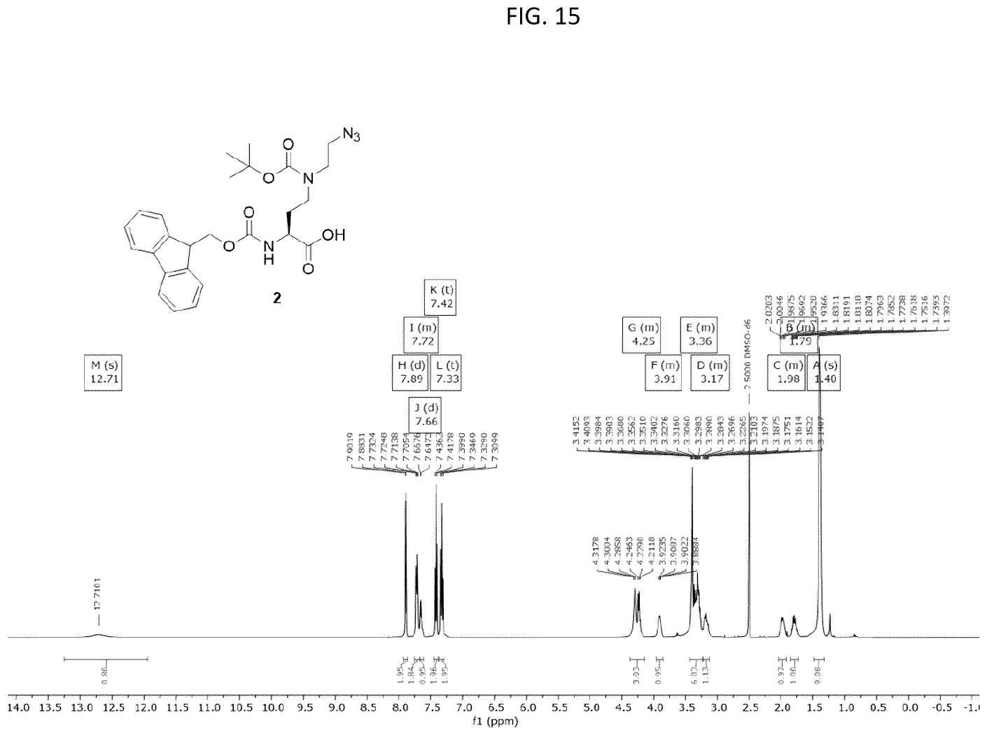

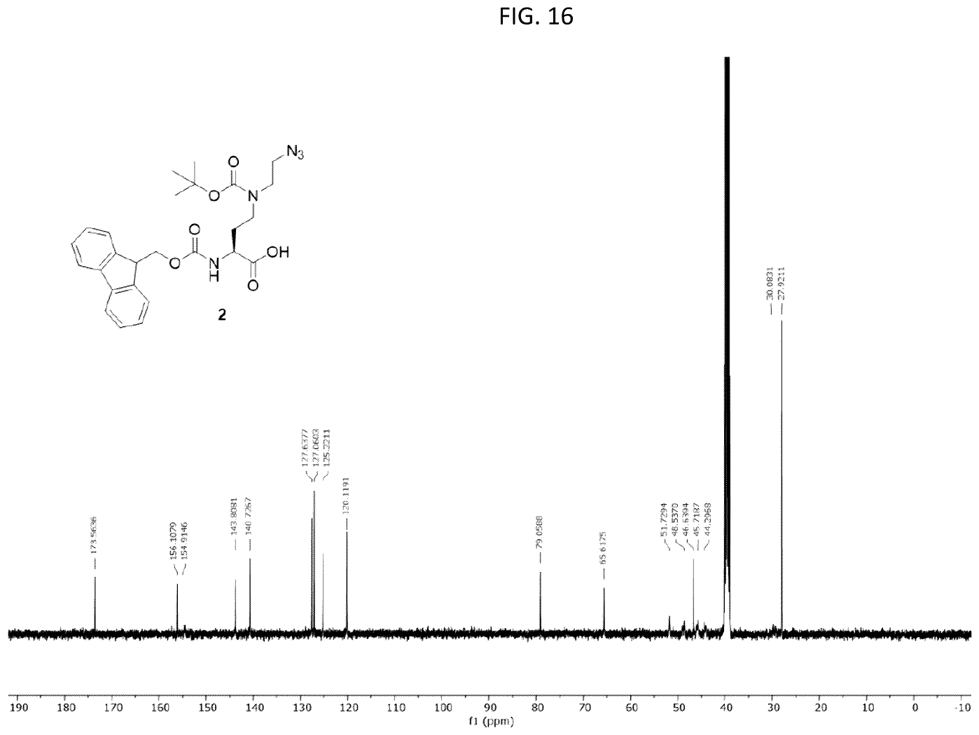

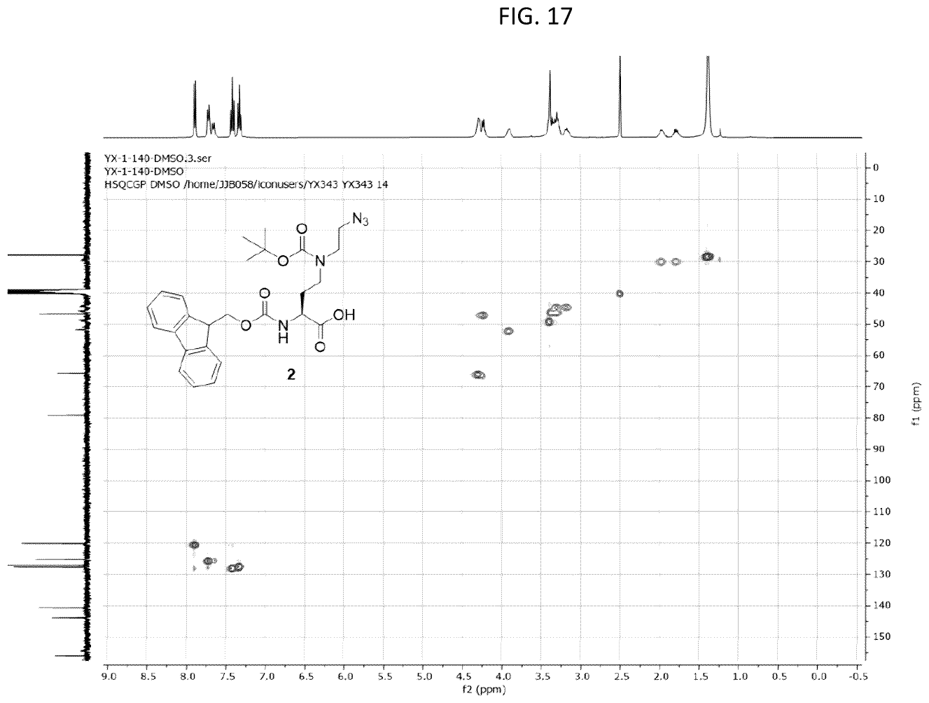

All reactions were conducted in flame dried round-bottom flasks. Automated column chromatography was performed on a Teledyne Isco Combiflash Rf using RediSep Rf Gold® C18 Reversed Phase Columns (20-40 μm). Commercially available chemicals and solvents were purchased from Sigma-Aldrich, Millipore Sigma, Combi-Blocks, Ambeed, Fisher Scientific, TCI Chemicals, and BroadPharm. All reagents and solvents were used as received without additional treatment or purification unless specified. Deuterated solvents were purchased from Sigma-Aldrich and Cambridge Isotope. A Bruker AV400 was used to record NMR spectra using residual solvent peak as reference. Chemical shifts are reported in ppm, and spin-spin coupling constants, J, are reported in Hz. Multiplicities are reported as follow: singlet (s), doublet (d), triplet (t), quartet (q), multiplet (m). NMR data is provided in FIGS. 11-17. High resolution mass spectra were obtained using an Agilent 6520 Accurate-Mass Q-Tof LC/MS system. Peptide synthesis was performed on a CEM Liberty Blue™ microwave-assisted peptide synthesizer (CEM, Charlotte, NC) unless specified as manual synthesis. RP-HPLC was performed with a solvent system of 0.1% TFA in Milli-Q H₂O (Standard A) and 0.1% TFA in 9:1 CH₃CN/H₂O (Standard B). Analytical RPHPLC was performed on an Agilent 1200 series HPLC, using a Vydac 218TP C18 column (250×4.6 mm, 5 μm), with a linear gradient from 0 to 100% B at a rate of 1% B per min, at a flow rate of 0.25 mL/min and a column temperature of 40° C. Preparative RP-HPLC was performed on a Waters 600 system, equipped with a Waters 2489 UV detector and a Vydac 218TP C18 column (250×22 mm, 10 μm), at a flow rate of 8.0 mL/min and a column temperature of 40° C. Gradients for preparative RP-HPLC and elution profiles for each specific peptide are described below. Analytical LCMS was performed on a Shimadzu LCMS 2020, using a Luna C18 column 100 Å (150×3 mm, 5 μm), with a linear gradient from 0 to 90% B at a rate of 2% B per min and a column temperature of 40° C. Solvents for LCMS consisted of 0.1% Formic acid in H₂O (Standard A) and 0.1% Formic acid in CH₃CN (Standard B).

### Synthesis of Amino Acid 2

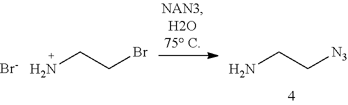

### 2-azidoethan-1-amine (4)

A round bottom flask was charged with 2-bromoethylamine hydrobromide (2.0 g, 9.8 mmol, 1 equiv.), sodium azide (1.9 g, 29 mmol, 3 equiv.) and water (28 mL, 0.35 M). After stirring at 75° C. overnight, the resulting mixture was cooled down, extracted with diethyl ether (50 mL*3), washed with brine, dried over sodium sulfate, and concentrated by rotary evaporation on an ice bath. The title compound was obtained as a colorless oil (0.49 g, 5.6 mmol, 58%) and used in the next step without further purification. ¹H NMR (400 MHz, CDCl3) δ 3.36 (t, J=5.7 Hz, 2H), 2.88 (d, J=5.7 Hz, 2H). ¹³C NMR (101 MHz, CDCl3) δ 54.8, 41.5.

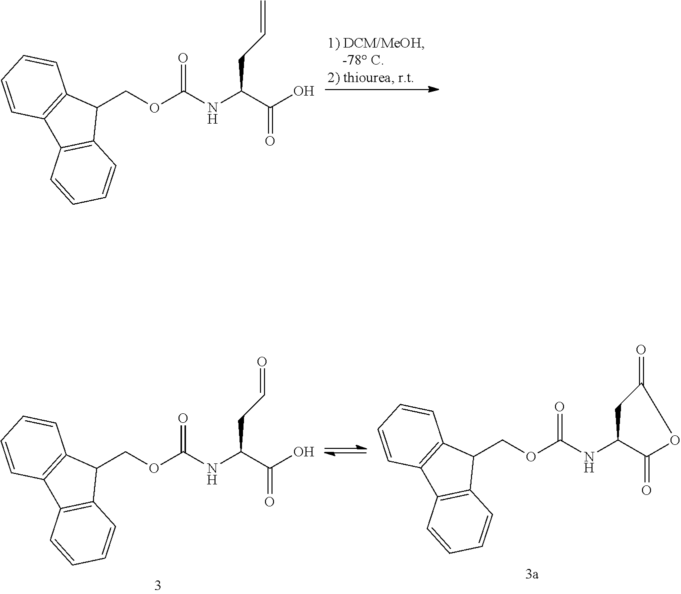 [SMILES: C=CC[C@H](NC(=O)OCC1c2ccccc2-c2ccccc21)C(=O)O ; O=CC[C@H](NC(=O)OCC1c2ccccc2-c2ccccc21)C(=O)O ; O=C1C[C@H](NC(=O)OCC2c3ccccc3-c3ccccc32)C(=O)O1]

### (S)-2-((((9H-fluoren-9-yl)methoxy)carbonyl)amino)-4-oxobutanoic acid (3, 3a

A round bottom flask was charged with (S)-2-((((9H-fluoren-9-yl)methoxy)carbonyl)amino)-pent-4-enoic acid (3.0 g, 8.9 mmol, 1 equiv.), anhydrous CH2C12 (44 mL, 0.2 M), and anhydrous MeOH (5 mL). At −78° C., O³ was bubbled into the mixture for 2 h. The resulting mixture was purged with nitrogen at −78° C. for 15 min to remove excessive O³, and then thiourea (0.76 mg, 9.8 mmol, 1.1 equiv.) was added. After stirring at room temperature for 2 h, the resulting mixture was diluted with CH₂Cl₂, washed with saturated ammonium chloride, water, and brine, dried over sodium sulfate, and concentrated by rotary evaporation. The title compound was obtained as a white solid (3.1 g, 9.0 mmol, quantitative) and used in the next step without further purification. ¹H NMR (400 MHz, MeOD) δ 7.79 (d, J=7.5 Hz, 2H), 7.72-7.56 (m, 2H), 7.39 (t, J=7.5 Hz, 2H), 7.31 (t, J=7.4 Hz, 2H), 4.74-4.57 (m, 1H), 4.58-4.28 (m, 3H), 4.27-4.04 (m, 1H), 2.43-1.83 (m, 2H). ¹³C NMR (101 MHz, MeOD) δ 175.7, 158.6, 145.2, 142.6, 128.8, 128.2, 126.2, 120.9, 97.1, 68.1, 52.4, 39.6, 39.3. HRMS [M+H]+m/z calc. for [C₁₇H₁₈O₅N]+340.1185, found 340.1181.

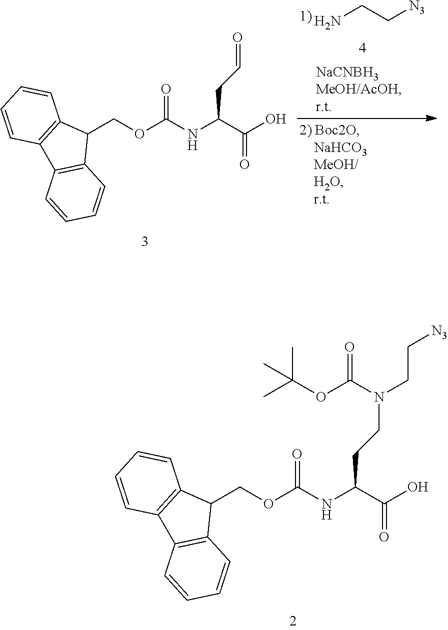 [SMILES: [N-]=[N+]=NCCN ; O=CC[C@H](NC(=O)OCC1c2ccccc2-c2ccccc21)C(=O)O ; CC(C)(C)OC(=O)N(CCN=[N+]=[N-])CC[C@H](NC(=O)OCC1c2ccccc2-c2ccccc21)C(=O)O]

### (S)-2-((((9H-fluoren-9-yl)methoxy)carbonyl)amino)-4-((2-azidoethyl)(tertbutoxycarbonyl)-amino)butanoic acid (2

A dry round bottom flask was charged with 2-azidoethan-1-amine 4 (1.35 g, 15.7 mmol, 2 equiv.), anhydrous MeOH (16 mL, 0.5 M) and glacial acetic acid (1.80 mL, 31.4 mmol, 4 equiv.). At 0° C., sodium cyanoborohydride (1.23 g, 19.6 mmol, 2.5 equiv.) and (S)-2-((((9H-fluoren-9-yl)methoxy)carbonyl)amino)-4-oxobutanoic acid 3 (2.66 g, 7.84 mmol, 1 equiv.) was added slowly to the flask. The resulting mixture was stirred under nitrogen at room temperature for 2 h. At 0° C., water (16 mL), sodium bicarbonate (7.90 g, 94.0 mmol, 12 equiv.) and di-tert-butyl decarbonate (3.76 g, 17.2 mmol, 2.2 equiv.) was added slowly. After stirring at room temperature for 4 h, the resulting mixture was concentrated by rotary evaporation, acidified by 1 M HCl, extracted with EtOAc three times. All organic layers were combined, washed with water and brine, dried sodium sulfate, and concentrated by rotary evaporation. The title compound was obtained as a white solid (2.40 g, 4.69 mmol, 60%) after reverse phase column chromatography with C18 silica gel (H₂O (0.05% formic acid: CH₃CN 100:0 to 35:65). ¹H NMR (400 MHz, DMSO) δ 12.71 (s, 1H), 7.89 (d, J=7.5 Hz, 2H), 7.81-7.68 (m, 2H), 7.66 (d, J=8.1 Hz, 1H), 7.42 (t, J=7.4 Hz, 2H), 7.33 (t, J=7.4 Hz, 2H), 4.50-4.16 (m, 3H), 3.98-3.79 (m, 1H), 3.45-3.23 (m, 5H), 3.24-3.05 (m, 1H), 2.03-1.92 (m, 1H), 1.88-1.68 (m, 1H), 1.40 (s, 9H). ¹³C NMR (101 MHz, DMSO) δ 173.6, 156.1, 154.9, 143.8, 140.7, 127.6, 127.1, 125.2, 120.1, 79.1, 65.6, 51.7, 48.5, 46.6, 45.7, 44.3, 30.08, 27.9. HRMS [M+H]+m/z calc. for [C₂₆H₃₂O₆N₅]⁺510.2353, found 510.2339.

### Peptide Synthesis

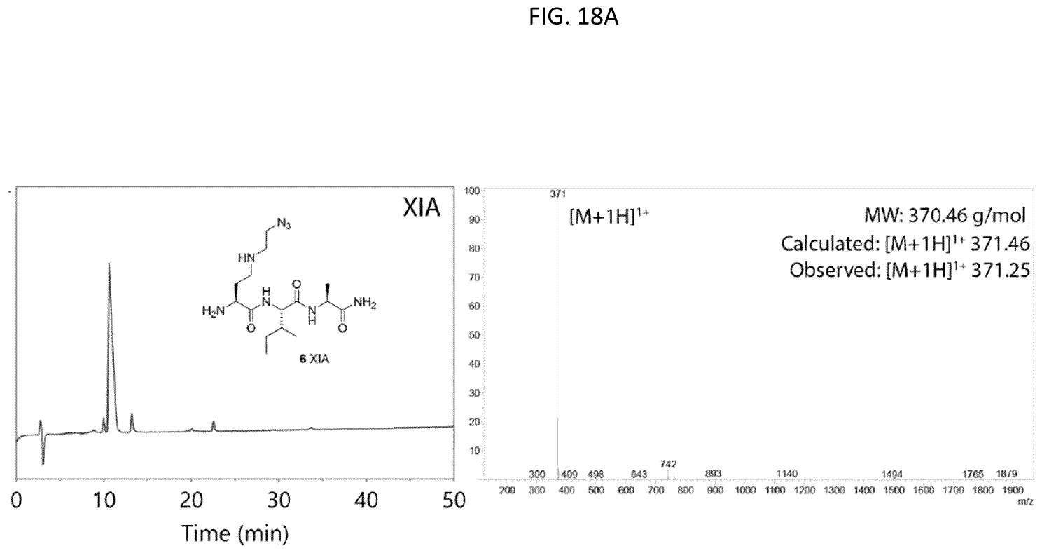

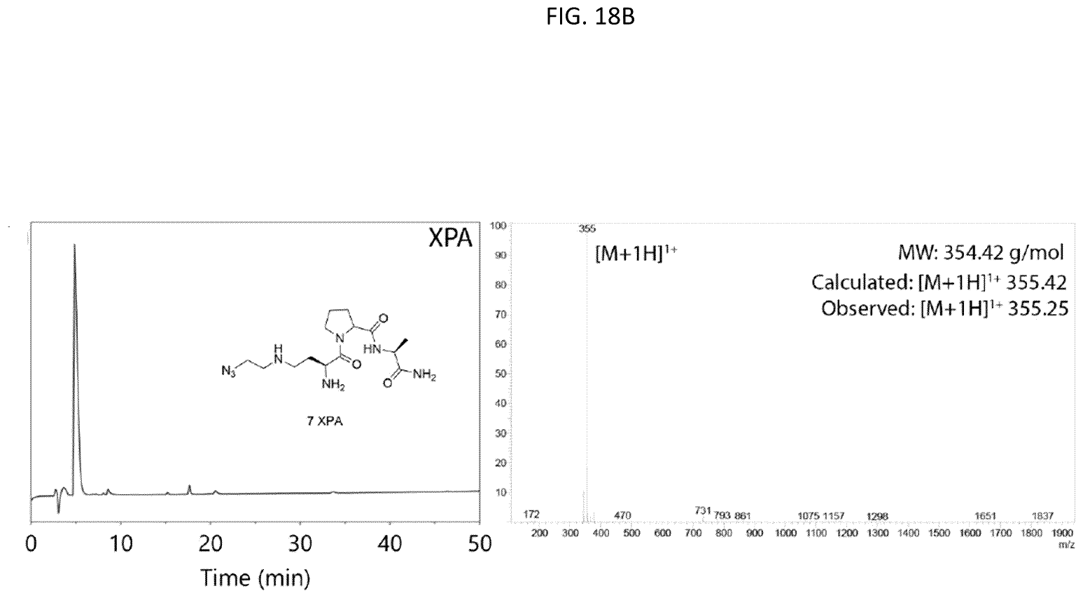

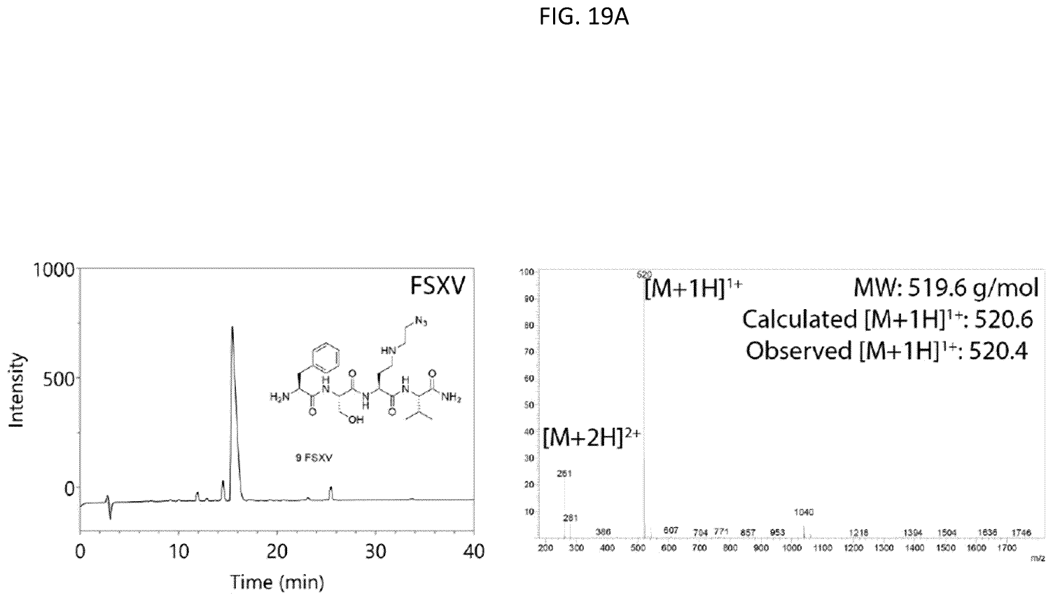

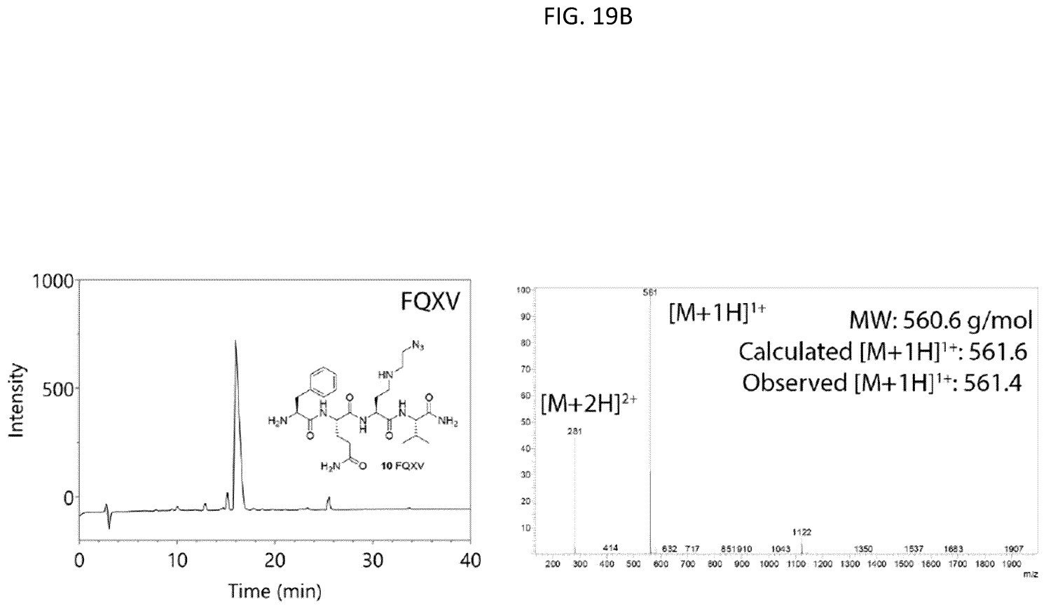

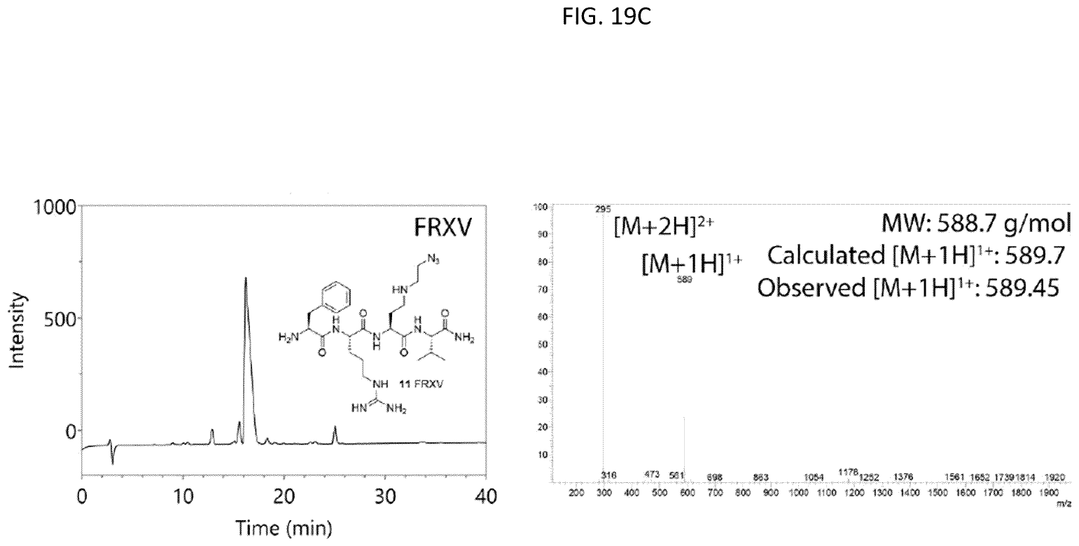

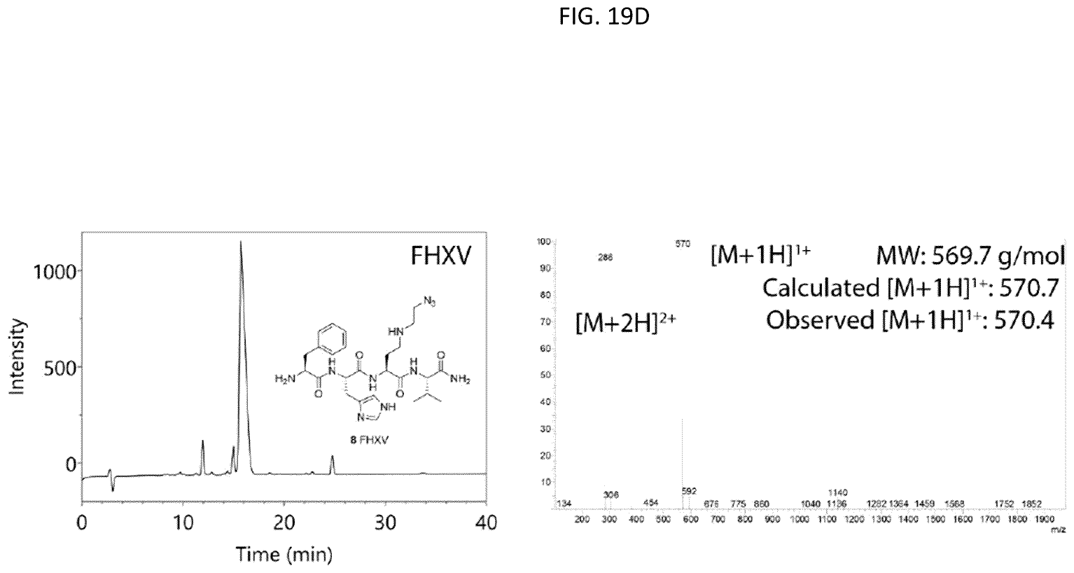

Tripeptides and tetrapeptides 6-11 were synthesized manually by Fmoc solid phase peptide synthesis using ProTide Rink amide resin (loading: 0.58 mmol/g) at 0.05 mmol scale. Peptide 9 synthesis by coupling condition 4 (Oxyma/DIC) was performed in a microwave-assisted Liberty Blue at 0.1 mmol scale.
Fmoc deprotection: Fmoc deprotection was carried out with 20% piperidine in DMF at room temperature twice per cycle (4 mL, 5 min). After the deprotection, the resin was washed with DMF 5 times.
Coupling condition 1: 4 equivalents of amino acids (0.2 M in DMF) were pre-activated with 4 equivalents of HATU (0.5 M in DMF) and 6 equivalents of DIPEA (0.75 M in DMF) for 2 min. Coupling solution was added to the resin and shaken for 45 min. Coupling completion was assessed by Kaiser Test. Resin was washed 3 times with DMF and 1 time with DCM before the next cycle.
Coupling condition 2 (HOAt, DIC): 5 equivalents of amino acids were pre-activated with 5 equivalents of HOAt in DMF and 5 equivalents of DIC for 20 min. Coupling solutions were added to the resin and shaken for 1 h. Coupling completion was assessed by Kaiser Test. Resin was washed 3 times with DMF and 1 time with DCM before the next cycle.
Coupling condition 3 (PyBOP, DIPEA): 5 equivalents of amino acids were pre-activated with 5 equivalents of PyBOP in DMF and 10 equivalents of DIPEA for 2 min. Coupling solutions were added to the resin and shaken for 1 h. Coupling completion was assessed by Kaiser Test. Resin was washed 3 times with DMF and 1 time with DCM before the next cycle.
Coupling condition 4 (Oxyma, DIC): 2.5 mL of amino acid solution (0.2 M in DMF), 0.5 mL of Oxyma solution (1 M in DMF), and 1 mL of DIC solution (0.5 M in DMF) were added to the resin. The reaction was heated at 90° C. for 4 min.
Cleavage from resin: Peptides were cleaved from the dried resin (0.05 mmol) with 5 mL of cleavage cocktail (95:2.5:2.5) TFA, triisopropylsilane, and water. After stirring at room temperature for 3 h, TFA was removed by argon gas flushing at room temperature. Crude peptide was precipitated with cold diethyl ether followed by two washes, dissolved in water, and lyophilized before purification.
   Peptides 6-11 were analyzed by analytical RP-HPLC and LCMS as the crude product.
   Chromatograms and mass spectroscopy data are provided in FIGS. 18-19.

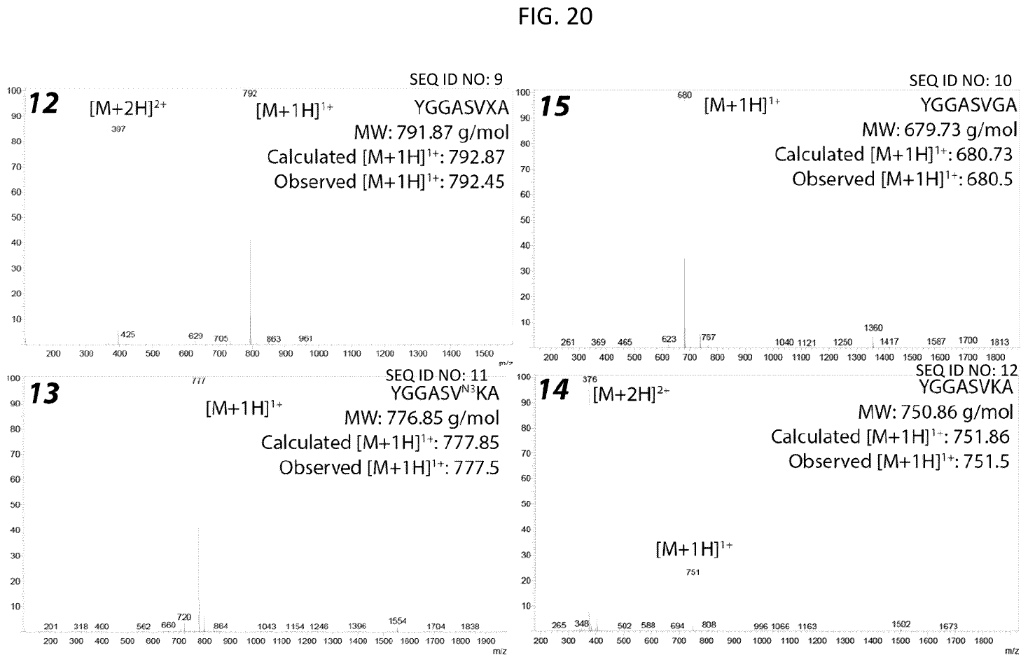

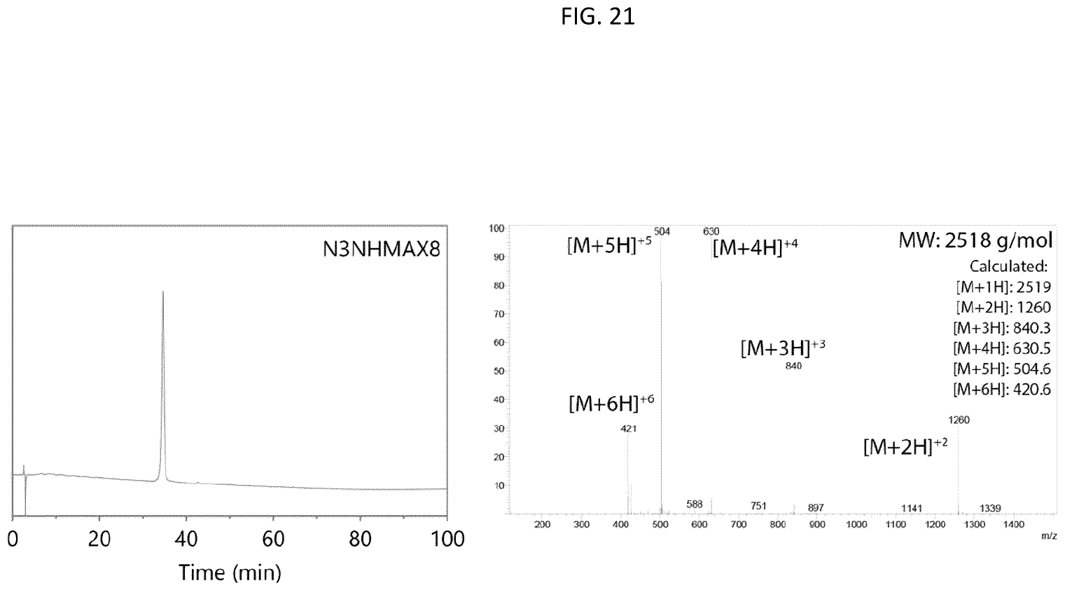

Peptides 12-18 were synthesized using a CEM Liberty Blue microwave-assisted peptide synthesizer and rink amide resin (loading: 0.58 mmol/g) at 0.1 or 0.25 mmol scale.
Fmoc deprotection: Fmoc deprotection was carried out with 4 mL of 20% piperidine in DMF using a standard microwave deprotection method (15 s at 75° C. (155W)) followed by 50 s at 90° C. (30W). After the deprotection, the resin was washed with DMF 4 times for 5 min each wash.
Coupling condition (Oxyma, DIC): 2.5 mL of amino acid solution (0.2 M in DMF), 0.5 mL of Oxyma solution (1 M in DMF), and 1 mL of DIC solution (0.5 M in DMF) were added to the resin. The reaction was heated at 90° C. for 4 min.
Cleavage from resin: Peptides were cleaved from the dried resin (0.1 mmol) with 10 mL of cleavage cocktail (95:2.5:2.5) TFA, triisopropylsilane, and water. After stirring at room temperature for 3 h, TFA was removed by argon gas flushing at room temperature. Crude peptide was precipitated with cold diethyl ether followed by two washes, dissolved in water, and lyophilized before purification.
HPLC purification of peptide: Crude peptide was purified by preparative RP-HPLC and lyophilized to afford pure peptide. Specific gradients and elution profiles are provided below. Chromatograms and mass spectroscopy data are provided in FIGS. 20-21.

Peptide 12: Preparative RP-HPLC gradient: 0% STD B for 7 min followed by a linear gradient to 100% STD B at 0.5% STD B/min. Peptide elutes at 15% STD B. RP-HPLC chromatogram is shown in FIG. 20.

Peptide 13: Preparative RP-HPLC gradient: 0% STD B for 2 min followed by a linear gradient to 4% STD B at 1.33% B/min, followed by a linear gradient to 100% STD B at 0.5% B/min. Peptide elutes at 20% STD B.

Peptide 14 and 15 were analyzed by analytical RP-HPLC as the crude.

Peptide 16: Preparative HPLC gradient: 0% STD B for 2 min followed by a linear gradient to 19% STD B at 6.33% B/min, then a linear gradient to 30% STD B at 0.46% B/min and an isocratic gradient at 30% STD B for 5 min, and linear gradient to 100% B at 0.5% B/min.

Peptide 17: Insoluble powder in water and acetonitrile. It is soluble in Trifluoroethanol.

Peptide 18 or MAX8 previously described (Haines-Butterick et al., Controlling hydrogelation kinetics by peptide design for three-dimensional encapsulation and injectable delivery of cells. Proc. Natl. Acad. Sci. USA, 2007, 104 (19), 7791-7796).

### Kinetic Experiments Between DBCO and Peptide 12 or 13

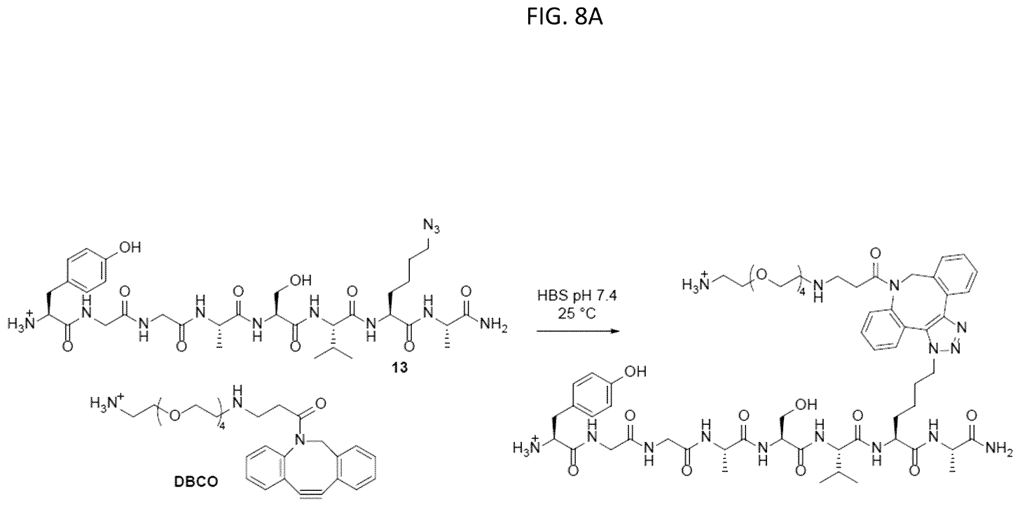

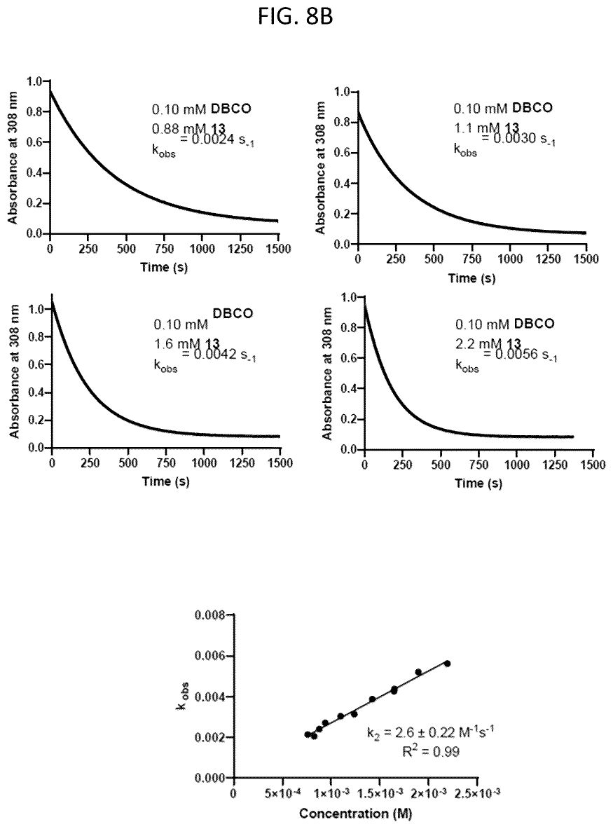

Solutions of 3.6, 7.1, 11, 14 mM peptide 12 and 0.88, 1.1, 1.6, 2.2 mM peptide 13 were prepared in HBS pH 7.4. Exact concentration of peptide was determined by the absorbance of peptide at 280 nm on UV-Vis spectrometer. Solution of 10 mM DBCO was prepared in DMSO. The reactions between peptide and DBCO were measured under pseudo-first order conditions using UV-Vis spectrophotometer. Solutions of 1 μL DBCO and 100 μL peptide were mixed in a cuvette at 25° C. The reaction was monitored by the decay of absorbance associated with the DBCO at maximum absorbance wavelength 308 nm. Reactions were repeated in duplicate. With Prism software, an observed rate constant, k_{obs}, was obtained by nonlinear regression. Second order rate constants, k₂, were calculated by linear regression between k_{obs} and concentration of peptide. Data is presented in FIGS. 7-8.

### Kinetic Experiments Between BCN and Amino Acid 2 or Azido Lysine

Solutions of BCN and amino acids were prepared in MeOD. The reaction between BCN and amino acids was measured under second order conditions using NMR. Solutions of BCN and amino acids were mixed in NMR tubes, resulting in 22 mM initial concentration of BCN ([A]0) and 20 mm initial concentration of amino acid ([B]0). The reaction was monitored by ¹H NMR. Concentration of product ([P]) at each time point was determined by the integration of red proton in the product using blue proton as internal standard. The value of k₂t was calculated by equation 1. With Prism software, second order rate constants, k₂, was calculated by linear regression between k2t and time. Reactions were repeated in duplicate. Data is resented in FIGS. 9-10.

$${k}_{2} t = \frac{1}{{[ B ]}_{0} - {[ A ]}_{0}} \times ln \frac{{[ A ]}_{0} ( {[ B ]}_{0} - [ P ] )}{{( {[ A ]}_{0} - [ P ] ) [ B ]}_{0}} \qquad ( Equation 1 )$$

N3NHMAX8 (16) Reaction with BCN Conditions

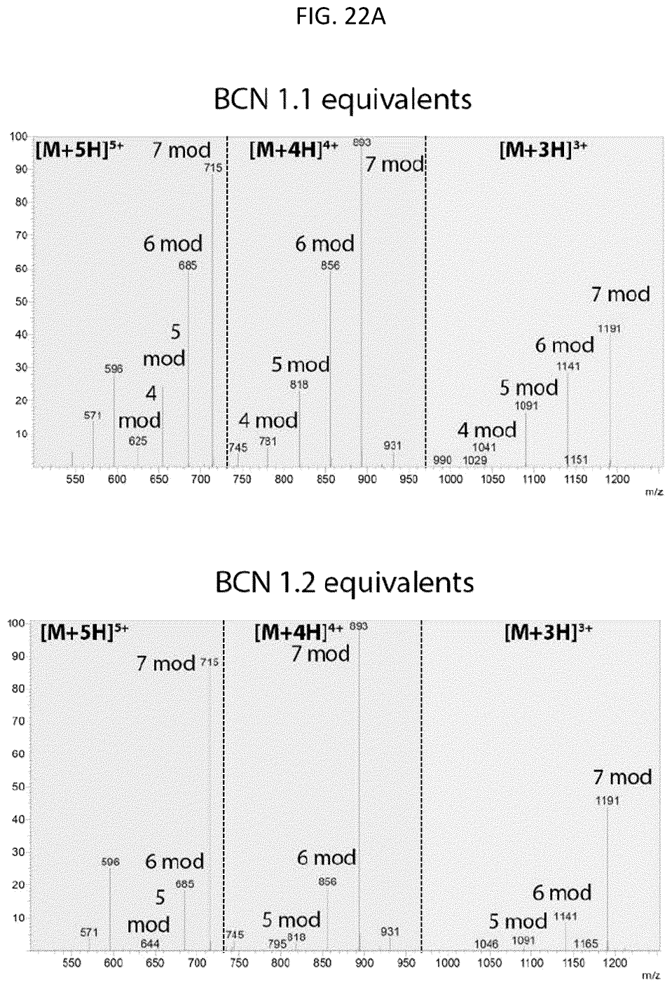

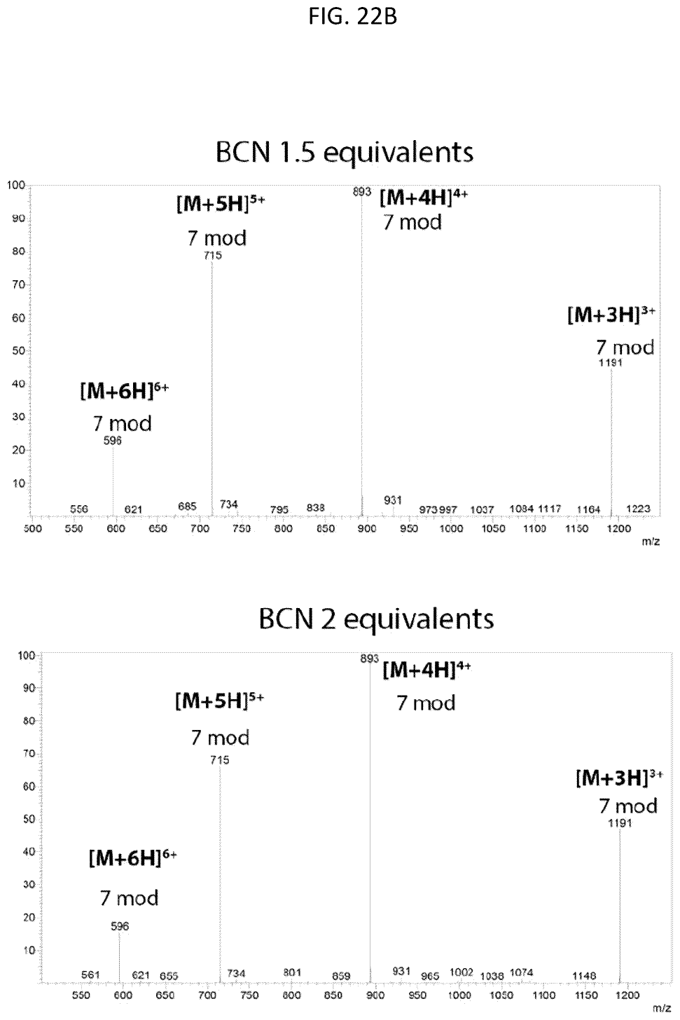

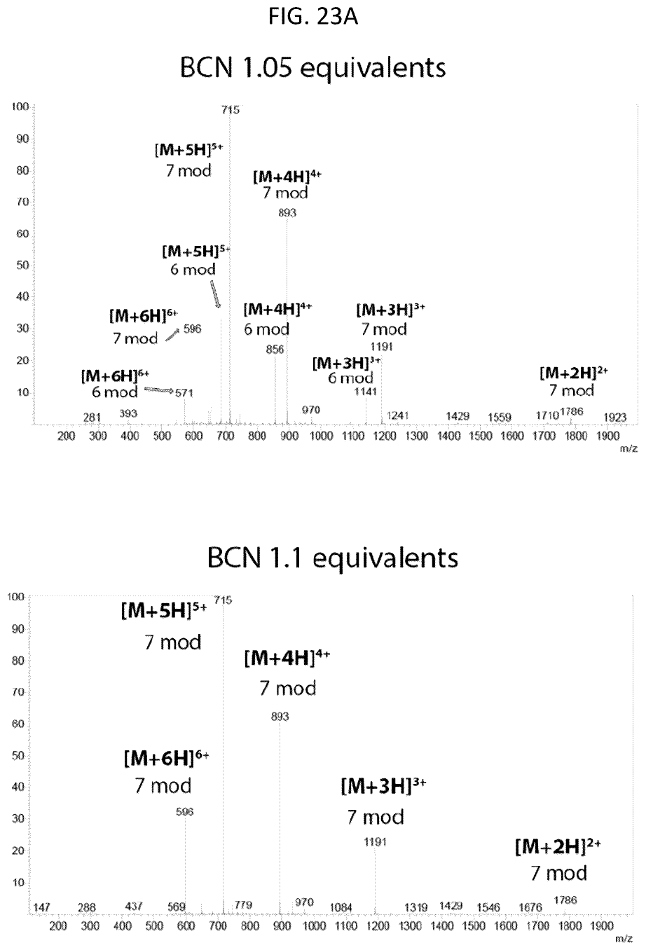

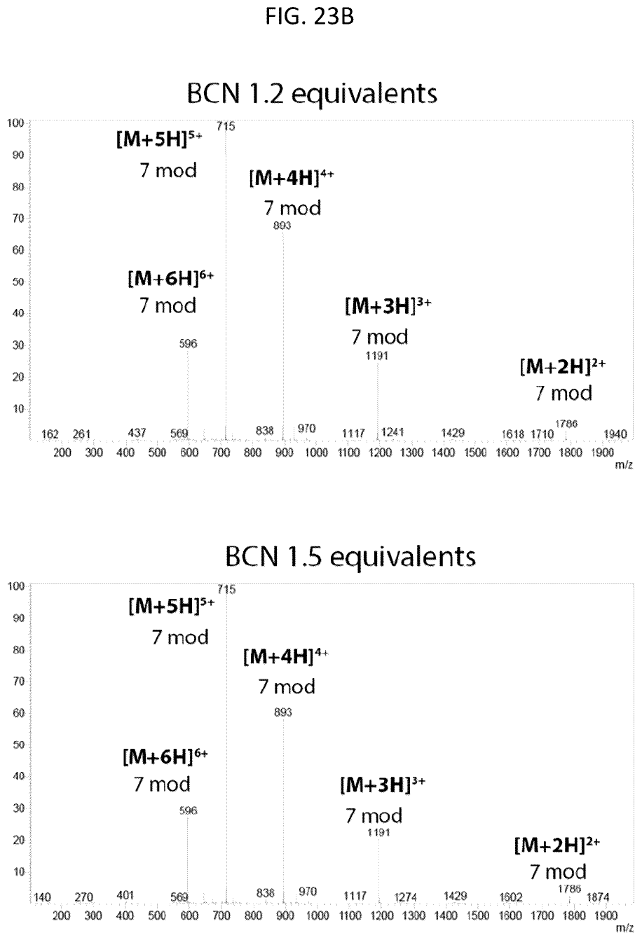

5 mM solution of peptide 16 was prepared in water. Stock solutions of BCN were prepared in DMSO at 350, 262.5, 210, 192.5, 183.75 mM for reactions with 2, 1.5, 1.2, 1.1, and 1.05 equivalent, respectively. 50 μL of peptide solution were mixed with 40 μL water and 10 μL BCN stock solutions. Reaction mixtures were stirred for 24 h at room temperature or 37° C. After 24 h, reaction mixtures were centrifuged for 1 min at 12000 rpm to pellet remaining BCN and diluted 1:10 with acetonitrile for LCMS and HPLC characterization. Complete mass spectroscopy data showing all observed charge states are provided in FIGS. 22-23.

### Example 2

### β-Hairpin Peptides Peptide Hydrogels Containing the Unnatural Lysine

This example provides a series of β-hairpin peptides containing the hydrophilic azide-containing lysine residue described in Example 1.

Solid-phase peptide synthesis was used to prepare β-hairpin peptides containing the unnatural lysine residue described in Example 1. The sequence of the β-hairpin peptides is as follows:

| | XP1 |
|---|---|
| (SEQ ID NO: 1) | |
| | Ac-VLTZVZTZV^{D}PPTZVQVZVFV-NH₂ |
| | XP2 |
| (SEQ ID NO: 2) | |
| | VZTZVEVEV^{D}PPTEVETZVZV-NH₂ |
| | XP3 |
| (SEQ ID NO: 3) | |
| | VETZVEVZV^{D}PPTEVZTEVZV-NH₂ |
| | XP4 |
| (SEQ ID NO: 3) | |
| | VETZVEVZV^{D}PPTEVZTEVZV |
| | XP5 |
| (SEQ ID NO: 2) | |
| | Ac-VZTZVEVEV^{D}PPTEVETZVZV |
| | XP6 |
| (SEQ ID NO: 3) | |
| | Ac-VETZVEVZV^{D}PPTEVZTEVZV |
| | XP7 |
| (SEQ ID NO: 4) | |
| | VZVZVZVZV^{D}PPTZVZVZVZV-NH₂ |
| | XP8 |
| (SEQ ID NO: 5) | |
| | VZVZVZVZV^{D}PPTZVEVZVZV-NH₂ |

wherein each Z is the unnatural lysine set forth as:

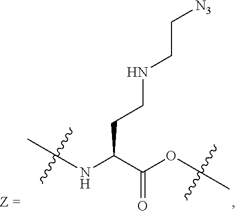 [SMILES: *N[C@@H](CCNCCN=[N+]=[N-])C(=O)O*]

Ac indicates that the peptide is acetylated at the N-terminus, and NH₂ indicates that the peptide is amidated at the C-terminus. The XP8 peptide is also referred to as N3NHMAX8 peptide or azido-MAX8 peptide.

The peptides were produced and purified according to standard solid-phase peptide synthesis methods. Peptide folding was assessed by CD measurement and the corresponding peptide hydrogel was assessed using oscillatory rheology.

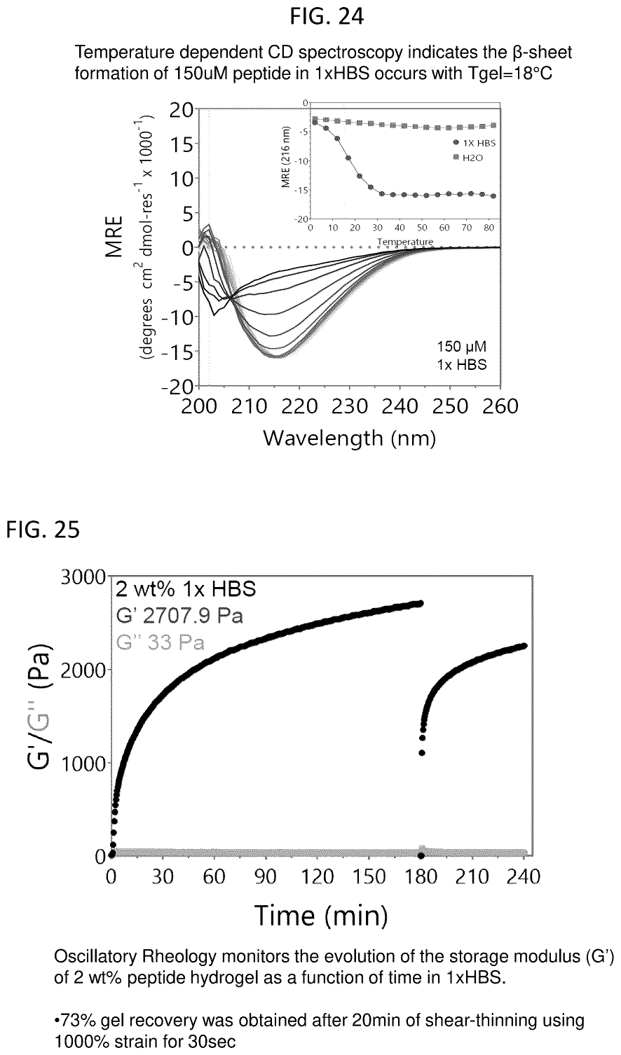

CD measurement of XP8 peptide demonstrated random coil in water, but in physiological buffer, there was β-sheet structure from the increased negative ellipticity peak centered around 216 nm (FIG. 24). The β-sheet formation of this peptide was temperature dependent, with 37° C. achieving maximum β-sheet structure (FIG. 24, inset).

The mechanical properties of the XP8 peptide hydrogel were further ascertained using rheology (FIG. 25). This peptide undergoes gelation in physiological buffer, and has shear-thinning recovery properties similar to previously reported cationic β-hairpin peptide hydrogels (see Haines-Butterick et al., Controlling hydrogelation kinetics by peptide design for three-dimensional encapsulation and injectable delivery of cells. Proc Natl Acad Sci USA 104, 7791-7796 (2007)). This shear-thinning recovery property potentially enables the peptide hydrogel to be used as a syringe-deliverable vehicle (see Yan et al. Injectable solid hydrogel: mechanism of shear-thinning and immediate recovery of injectable beta-hairpin peptide hydrogels. Soft Matter 6, 5143-5156, 2010).

Peptide Synthesis and Purification. Peptides were synthesized using standard Fmoc chemistry on an ABI 433A automated peptide synthesizer, using PL-Rink resin (0.25 mmol scale). Fmoc deprotection was performed using a cocktail of 1.0% DBU, 19% piperidine in NMP and monitored to completion by conductivity. Activation of 4 eq. of amino acid was achieved with 3.6 eq. HCTU and 10 eq DIEA in NMP, and the coupling proceeded for 30 min with constant vortexing. The peptide was cleaved from resin using 95% TFA, 2.5% TIPS, and 2.5% water for 3 hr, after which the resin was filtered and the collected filtrate was concentrated. Following ether precipitation, the crude solid was dried under vacuum. The peptides were purified by prep-HPLC. The crude peptide was dissolved at 2 mg/mL in Std C and 5 mL per run was injected into a Waters 600 system, equipped with a Waters 2489 UV Detector and a Phenomenex PolymerX RP-1 column (250×21.2 mm, 10 μm, heated to 40° C.). A gradient of 1% Std D per min was used for 25 min, followed by 0.5% Std D per min for 150 min. The UV trace at 220 nm was monitored and fractions containing the major peptide peak were combined and lyophilized. Purity was assessed by LCMS (Shimadzu LCMS 2020) and analytical HPLC (Agilent 1200 series, PolymerX RP-1 column, 250×4.6 mm, 10 μm) using gradients of 1% Std D per min. Purified peptide was converted to the sodium salt by dissolving at 1 mg/mL Std C and adding 1 eq NaOH per glutamate residue (5 eq NaOH per 1 eq peptide) using a 0.1 M NaOH stock. The solution was then frozen and lyophilized.

Circular dichroism (CD). CD spectroscopy of the peptides was measured on an Aviv 410 spectropolarimeter (Aviv Biomedical). A 300 μM solution of peptide was prepared and an equal volume of buffer (50 mM HEPES containing 300 mM NaCl, pH 7.4) was added. A peptide solution was quickly transferred to 1 mm path length cell previously equilibrated at 5° C. Then, CD wavelength spectra were collected from 200-260 nm at 37° C. CD spectra were also collected as a function of temperature to follow β-sheet formation at 216 nm (2-52° C. with a 5° C. increment and a 10 min equilibration time for each temperature point. The mean residue ellipticity [θ] was calculated from [θ]=(θ_{obs}/10lcr), where θ_{obs} is the measured ellipticity in millidegree, l is the cell path length in centimeters, c is the concentration in molar, and r is the number of residues of peptide sequence.

Oscillatory rheology. Rheology experiments were performed on an AR-G2 rheometer (TA Instruments) equipped with a 25-mm stainless steel parallel plate geometry with a 0.5 mm gap height. To mimic syringe delivery, a shear-thinning recovery procedure was used. A 2 wt % peptide solution in water was prepared on ice and an equal volume of buffer (50 mM HEPES containing 300 mM NaCl, pH 7.4) was added to initiate gelation. For the gelation by DMEM, a 1 wt % peptide in 285 mM sucrose was prepared and an equal volume of HEPES-supplemented DMEM was added. A 300 μL of the solution was immediately transferred to the rheometer plate equilibrated at 5° C. Oil was placed around the sample and on the plate to prevent evaporation. After the temperature was ramped to 37° C. (0.5° C. s⁻¹), a 1-hour dynamic time sweep was performed to monitor the storage (G′) and loss (G″) modulus of the resulting hydrogel. An angular frequency of 6 rad s⁻¹ and 0.2% strain was applied. After which, a 1000% strain was applied for 30 s to disrupt the material. Subsequently, the ability of the hydrogel to reheal itself was monitored by measuring the recovery of G′ at 6 rad s⁻¹ and 0.2% strain for additional 1 hour.

### Example 3

### Triiodo-Labeled β-Hairpin Peptide Hydrogel

This example describes labeling of the peptide hydrogel provided herein with a triiodo group for CECT imaging.

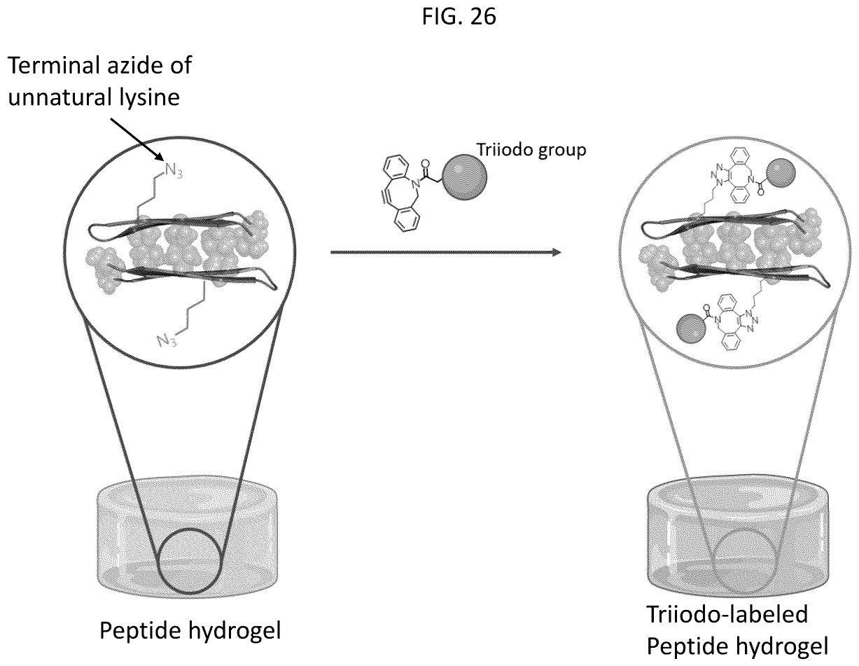

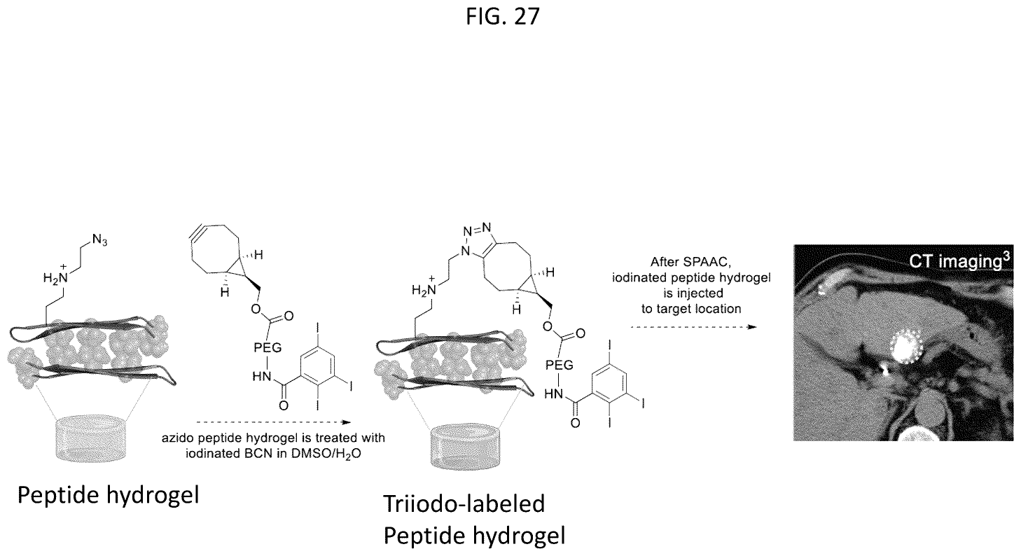

The general labeling strategy is illustrated in FIGS. 26 and 27. A peptide hydrogel formed with a β-hairpin peptide containing the unnatural lysine described herein (such as azido-MAX8 peptide hydrogel described in Example 2) is provided. The peptide hydrogel is incubated with a triiodo group compound linked (for example, via a PEG linker) to a copper-free SPAAC probe (such as BCN or DBCO) in a solution of DMSO and water. Under these conditions, the SPAAC probe ligates with the terminal azide of the unnatural lysine to link the triiodo group to the hydrogel, which can then be loaded in a syringe and delivered to a target location in a subject for CECT imaging.

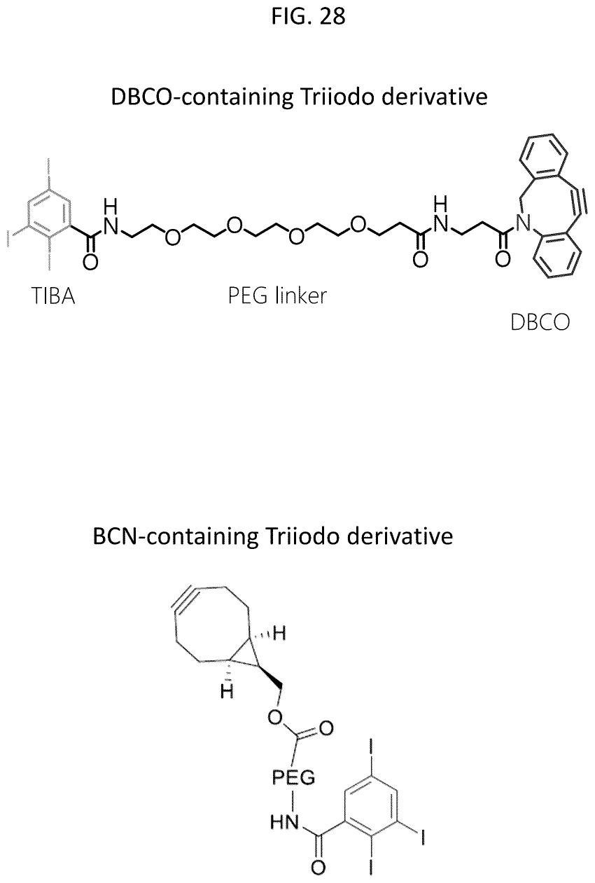

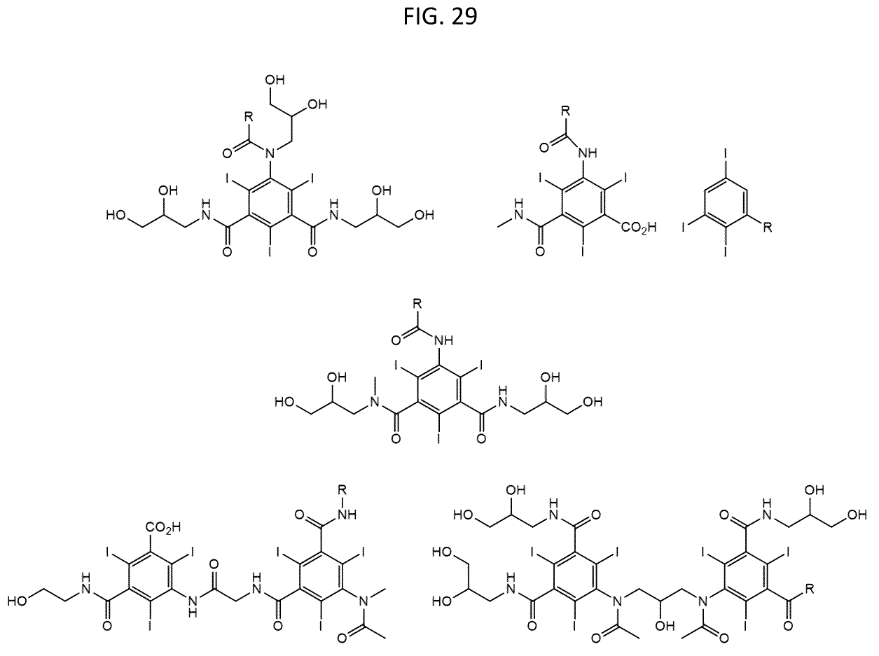

FIG. 28 shows exemplary DBCO- and BCN-containing triiodo derivatives for labeling of the peptide hydrogel formed with a β-hairpin peptide containing the unnatural lysine described herein. FIG. 29 shows different examples of triiodo-containing groups that can be used. These are linked to a SPAAC probe (such as DBCO or BCN) via a linker for labeling of the peptide hydrogel.

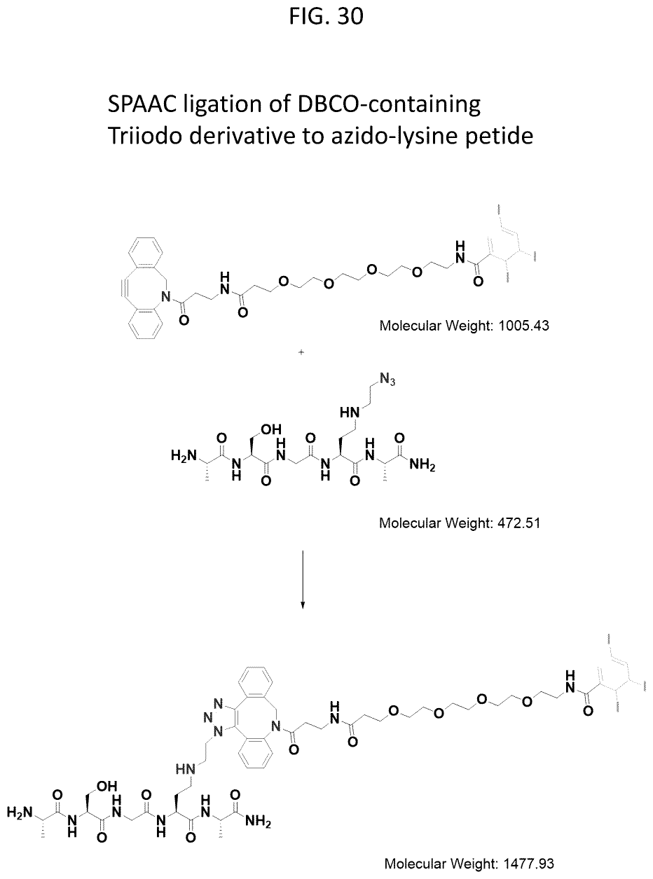

FIG. 30 illustrates ligation of a DBCO-containing triiodo derivative to a short peptide containing the azido-lysine described herein.

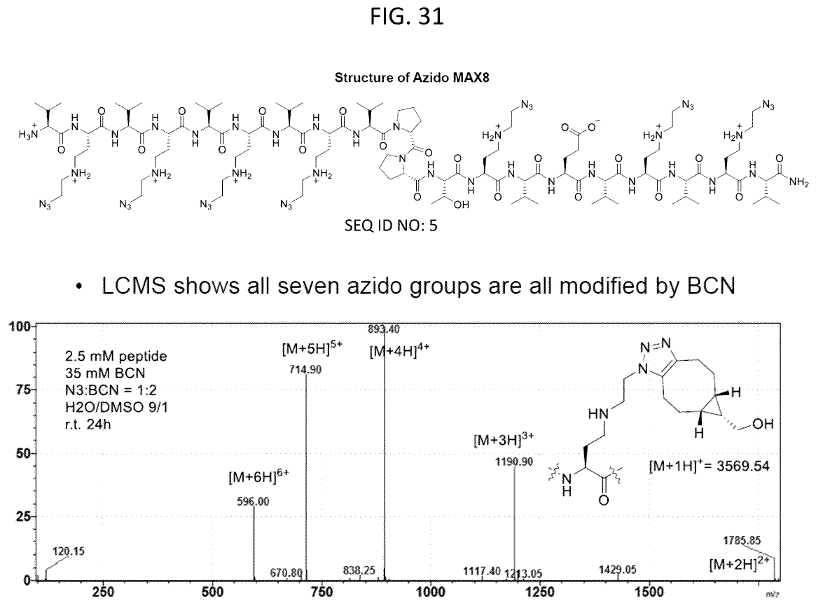

FIG. 31 provides data showing that SPAAC ligation of BCN to azido-MAX8 peptide. LCMS analysis demonstrated that the seven azido groups in azido-MAX8 are modified by BCN. The ligation conditions are shown in the inset.

The azide group of the unnatural lysine in the β-hairpin peptide of the peptide hydrogel is readily reacted with a SPAAC probe (e.g., BCN or DBCO) linked to a triiodo-containing molecule, such as Triiodobenzoic acid (TIBA), iohexol, iopromide, iothalamate, ioxaglate, and iodixanol. For example, the azide group of the unnatural lysine in the β-hairpin peptide of the peptide hydrogel is readily reacted with a SPAAC probe (e.g., BCN or DBCO) linked to any one of: the following for use as a CECT marker:

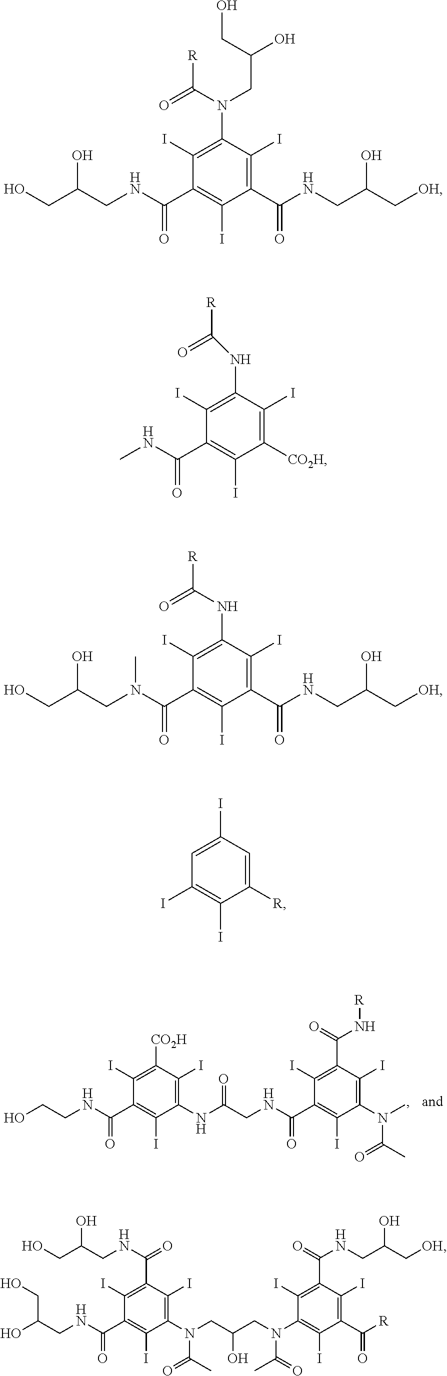

wherein R is the linkage to the unnatural lysine residues in the amphiphilic cationic β-hairpin peptide of the peptide hydrogel.

It will be apparent that the precise details of the methods or compositions described may be varied or modified without departing from the spirit of the described embodiments. We claim all such modifications and variations that fall within the scope and spirit of the claims below.

1. A peptide hydrogel formed from an amphiphilic cationic β-hairpin peptide, wherein the amphiphilic cationic β-hairpin peptide comprises an unnatural lysine according to Formula 2: 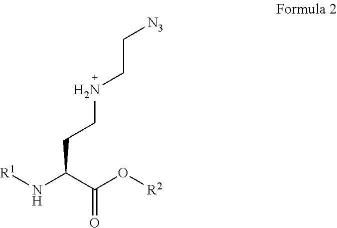
wherein R¹ is another amino acid in the peptide or the N-terminus of the peptide and R² is another amino acid in the peptide or the C-terminus of the peptide.

2. The peptide hydrogel of claim 1, wherein the amphiphilic cationic β-hairpin peptide comprises an amino acid sequence set forth as:

| (SEQ ID NO: 6) | |
|---|---|
| | (XZ)ₙ X^{D}PPX(ZX)ₙ; |
| (SEQ ID NO: 7) | |
| | (XZ)ₙ X^{D}PGX(ZX)ₙ; |
| | or |
| (SEQ ID NO: 8) | |
| | (XZ)ₙ XNGX(ZX)ₙ; |

and
wherein
each X is individually selected from V, I, L, M, T, F, W, and Y;
at least one Z is the unnatural lysine and the remaining Z are individually selected from any amino acid;
X^{D} is a D-amino acid selected from a V, I, L, M, T, F, W, and Y D-amino acid; and
n is from 3 to 5.

3. The peptide hydrogel of claim 2, wherein the amphiphilic cationic β-hairpin peptide comprises an amino acid sequence set forth as:

| (SEQ ID NO: 6) | |
|---|---|
| | (XZ)ₙ X^{D}PPX(ZX)ₙ; |

wherein
each X is individually selected from V, I, L, M, T, F, W, and Y;
at least one Z is the unnatural lysine and the remaining Z are individually selected from any amino acid;
X^{D} is a D-amino acid selected from a V, I, L, M, T, F, W, and Y D-amino acid; and
n is from 3 to 5.

4. The peptide hydrogel of claim 1, wherein the amphiphilic cationic β-hairpin peptide comprises or consists of an amino acid sequence set forth as any one of

| (SEQ ID NO: 1) | |
|---|---|
| | VLTZVZTZV^{D}PPTZVQVZVFV; |
| (SEQ ID NO: 2) | |
| | VZTZVEVEV^{D}PPTEVETZVZV; |
| (SEQ ID NO: 3) | |
| | VETZVEVZV^{D}PPTEVZTEVZV; |
| (SEQ ID NO: 4) | |
| | VZVZVZVZV^{D}PPTZVZVZVZV; |
| | and |
| (SEQ ID NO: 5) | |
| | VZVZVZVZV^{D}PPTZVEVZVZV; |

and
wherein each Z is the unnatural lysine.

5. The peptide hydrogel of claim 1, wherein the amphiphilic cationic β-hairpin peptide is acetylated at the N-terminus, amidated at the C-terminus, or acetylated at the N-terminus and amidated at the C-terminus.

6. The peptide hydrogel of claim 4, wherein the amphiphilic cationic β-hairpin peptide comprises or consists of:

| | XP1 |
|---|---|
| (SEQ ID NO: 1) | |
| | Ac-VLTZVZTZV^{D}PPTZVQVZVFV-NH₂ |
| | XP2 |
| (SEQ ID NO: 2) | |
| | VZTZVEVEV^{D}PPTEVETZVZV-NH₂ |
| | XP3 |
| (SEQ ID NO: 3) | |
| | VETZVEVZV^{D}PPTEVZTEVZV-NH₂ |
| | XP4 |
| (SEQ ID NO: 3) | |
| | VETZVEVZV^{D}PPTEVZTEVZV |
| | XP5 |
| (SEQ ID NO: 2) | |
| | Ac-VZTZVEVEV^{D}PPTEVETZVZV |
| | XP6 |
| (SEQ ID NO: 3) | |
| | Ac-VETZVEVZV^{D}PPTEVZTEVZV |
| | XP7 |
| (SEQ ID NO: 4) | |
| | VZVZVZVZV^{D}PPTZVZVZVZV-NH₂ |
| | XP8 |
| (SEQ ID NO: 5) | |
| | VZVZVZVZV^{D}PPTZVEVZVZV-NH₂ |

wherein each Z is the unnatural lysine, Ac indicates that the peptide is acetylated at the N-terminus, and NH₂ indicates that the peptide is amidated at the C-terminus.

7. The peptide hydrogel of claim 1, wherein one or more of the unnatural lysine residues in the amphiphilic cationic β-hairpin peptide are linked to a detectable marker by Strain Promoted Azide-Alkyne Cycloaddition (SPAAC) chemistry via the terminal azide group of the unnatural lysine.

8. The peptide hydrogel of claim 7, wherein the detectable marker comprises a contrast agent for contrast enhanced computed tomography (CECT) imaging.

9. The peptide hydrogel of claim 8, wherein the contrast agent for CECT comprises a triiodo group.

10. The peptide hydrogel of claim 9, wherein the contrast agent for CECT is selected from any one of Triiodobenzoic acid (TIBA), iohexol, iopromide, iothalamate, ioxaglate, and iodixanol.

11. The peptide hydrogel of claim 9, wherein the contrast agent for CECT comprises any one of: 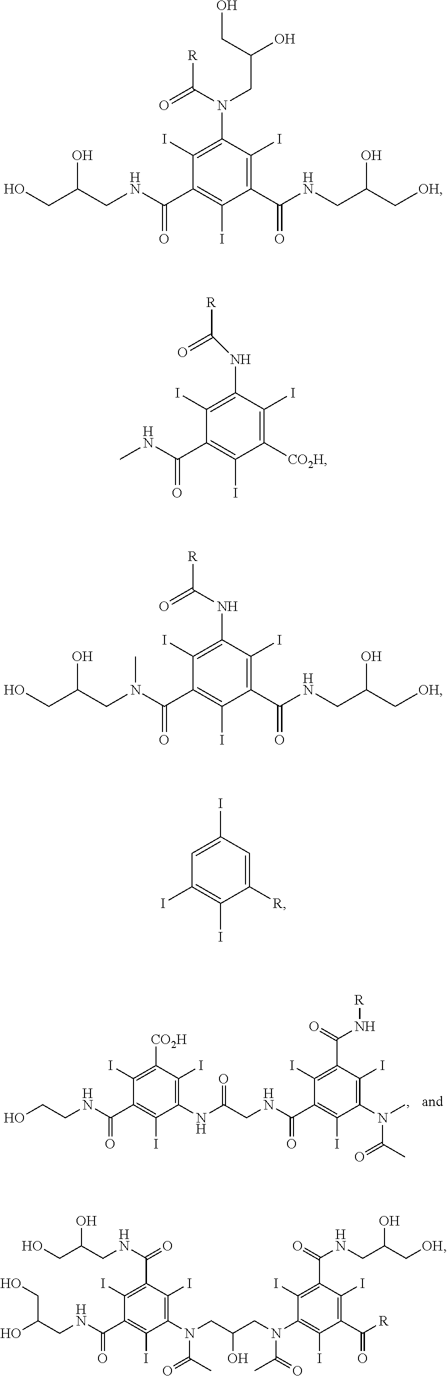
wherein R is a linkage to the unnatural lysine residues in the amphiphilic cationic β-hairpin peptide by SPAAC chemistry.

12. The peptide hydrogel of claim 1, wherein the hydrogel undergoes shear-thinning upon application of shear stress, and rheological recovery upon removal of the shear stress.

13. The peptide hydrogel of claim 1, comprising a storage modulus of greater than 40 Pascal in the absence of shear.

14. The peptide hydrogel of claim 1, comprising from about 10 mM to about 400 mM NaCl and a pH of from about 7.0 to about 9.0.

15. The peptide hydrogel of claim 14, comprising about 150 mM NaCl and a pH of about 7.4.

16. The peptide hydrogel of claim 1, comprising from about 0.25% to about 4.0% w/v of the amphiphilic β-hairpin peptide.

17. A syringe, containing the peptide hydrogel of claim 1.

18. A method of making the peptide hydrogel comprising a contrast agent for contrast enhanced computed tomography (CECT) imaging, comprising:
providing a peptide hydrogel formed from an amphiphilic cationic β-hairpin peptide, wherein the amphiphilic cationic β-hairpin peptide comprises an unnatural lysine according to Formula 2: 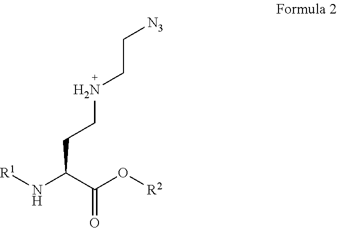
wherein R¹ is another amino acid in the peptide or the N-terminus of the peptide and R² is another amino acid in the peptide or the C-terminus of the peptide; and
incubating the peptide hydrogel with a contrast agent for CECT imaging linked to a strained difluorooctyne (DIFO) moiety under conditions sufficient for ligation of the strained DIFO moiety to the terminal azide of the unnatural amino acid;
thereby producing the peptide hydrogel comprising the contrast agent for CECT imaging.

19. The method of claim 18, wherein the strained DIFO moiety is bicyclononyne (BCN) or Dibenzocyclooctyne (DBCO).

20. The method of claim 18, wherein the contrast agent for CECT imaging is linked to the strained DIFO moiety by a PEG linker.

21. A method of contrast enhanced computed tomography (CECT) imaging of a subject, comprising administering an effective amount of the peptide hydrogel of claim 8 to a target location in the subject, and conducting a contrast CT scan of the target location.

22. An unnatural lysine residue, or a stereoisomer, tautomer, or pharmaceutically acceptable salt or ester thereof, according to Structure 1 or Formula I: 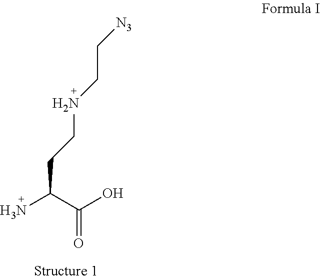
wherein, in Formula I, X¹ is a protecting group and X² is a protecting group.

23. The unnatural lysine residue of claim 22, wherein, in Formula 1, X¹ is fluorenylmethoxycarbonyl protecting group (Fmoc) and X² is tert-butyloxycarbonyl protecting (Boc) or (S)-2-((((9H-fluoren-9-yl)methoxy)carbonyl)amino)-4-((2-azidoethyl)((2-(trimethylsilyl)ethoxy)carbonyl)amino)butanoic acid (Teoc).

24. A polypeptide comprising the unnatural lysine residue of claim 22 linked by peptide bond to the polypeptide chain of the polypeptide.
